(19) United States
(12) Patent Application Publication
(10) Pub. No.: US 2005/0148598 A1
(43) Pub. Date: Jul. 7, 2005

(54) Trioxane derivatives

(76) Inventors: Paul Michael O'Neill, Liverpool (GB); Adrian Peter Higson, Lancashire (GB); Sara Taylor, Derbyshire (GB); Edward Irving, Cheshire (GB)

Correspondence Address: HEDMAN & COSTIGAN P.C., 1185 AVENUE OF THE AMERICAS, NEW YORK, NY, 10036, US

(21) Appl. No.: 10/497,731

(22) PCT Filed: Dec. 6, 2002
(86) PCT No.: PCT/GB02/05531

(30) Foreign Application Priority Data
Dec. 6, 2001 (GB) 0129215.0

(51) Int. Cl. A61K 31/496; A61K 31/355; C07D 493/14

(52) U.S. Cl. 514/254.11; 514/452; 544/375; 549/358

(57) ABSTRACT

The present invention relates to compounds of general formula (13), and pharmaceutically acceptable salts thereof, in which: n=an integer of from 1 to 4; A represents a trioxane-containing residue; B represents a group having the general formula: -D-E-F-, in which D is linked to A and represents an atom or group selected from the following (a, b, c, d), E represents a bivalent, optionally substituted organic radical; and F is linked to C and represents a group selected from the following: (e, f, g, h) and C represents a group containing at least two nitrogen atoms, (13).

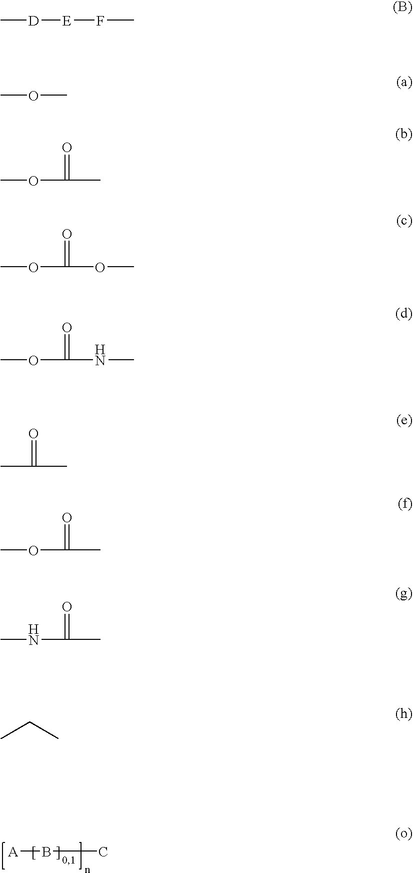

The present invention relates to certain compounds containing a trioxane moiety that have potent antimalarial activity and antitumour activity.

Artemisinin (1), which is also known as qinghaosu, is a tetracyclic 1,2,4-trioxane occurring in Artemisia annua. Artemisinin and its derivatives dihydroartemisinin (DHA) (2), artemether (3) and sodium artesunate (4) are used routinely in the treatment of malaria and have been found to be particularly effective against cerebral malaria.

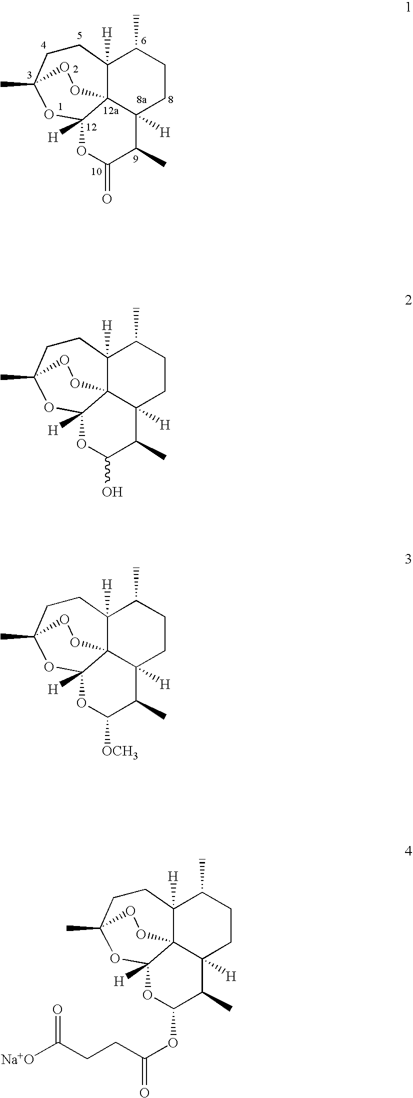

Different mechanisms of action have been proposed to account for the antimalarial action of artemisinin and its derivatives (Posner et al., J. Am. Chem. Soc. 1996, 118, 3537; Posner et al., J. Am. Chem. Soc. 1995, 117, 5885; Posner et al., J. Med. Chem. 1995, 38, 2273). Whilst the mechanism of action of artemisinin as an antimalarial has not been unequivocally established, it has been shown that the peroxide linkage is essential for antimalarial activity.

Certain artemisinin derivatives containing a peroxide moiety have also been tested for biological activity other than antimalarial activity. For example, the cytoxicity to Ehrlich ascites tumour cells of artemisinin, dihydroartemisinin, artemisitene, arteether, ethylperoxyartemisitene and an ether dimer of artemisinin has been demonstrated (Beekman et al., Phytother. Res., 1996, 10, 140; Woerdenberg et al., J. Nat. Prod., 1993, 56, 849).

Selective cancer cell cytoxicity from exposure to dihydroartemisinin and holotransferrin, a non heme iron transport protein saturated with iron, has also been disclosed (Lai et al., Cancer Lett., 1995, 91, 41 and U.S. Pat. No. 5,578,637), with the drug combination being approximately 100 times more effective on molt-4 cells than lymphocytes.

It is known that some biologically active molecules contain chemical groups which enable them to bind to DNA. The method by which DNA binding occurs depends upon the overall structure of the molecule and the nature of the chemical groups contained within the molecule. For instance, the major and minor grooves of the double helical DNA are occupied by water under physiological conditions. However, certain oligopeptidic compounds such as netropscin and disamycin can displace water molecules and form strong hydrogen bonds with hydrophilic groups along the DNA strands.

Alternatively, some compounds contain groups which are capable of intercalating with DNA. Intercalators are compounds which insert between the bases of DNA. Well characterised examples of intercalators are provided by anthracyclines, such as adriamycin and daunomycin, which are used for the treatment of cancer, and acridines, such as amascrine, which is used for treating acute leukaemia and malignant lymphomas. The antitumour activity is associated with the intercalating property of these compounds.

Naturally occurring polyamines, such as the tetra-amine spermine (5) and the triamine spermidine (6) occur in cells at micromolar concentrations, and may even rise to millimolar levels in certain cancer cells (Tabor & Tabor, Ann. Rev. Biochem, 1984, 53, 749). The biosynthetic building blocks for these and closely related polyamines are the alpha amino acids ornithine and lysine, affording the diamines putrescine (7) (1,4-diaminobutane) and cadaverine (8) (1,5-diamino pentane) respectively.

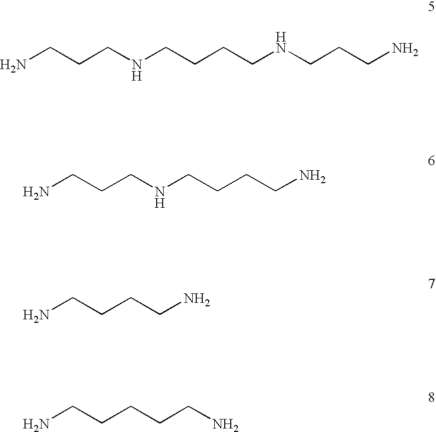

In recent years, it has been established that polyamines and polyamine amides have potential as novel therapeutic lead compounds in the design of anti-tumour agents. Other workers, (Bergeron et al., Med. Chem., 1987, 31, 1183, Bergeron et al., Cancer Res., 1989, 49, 2959) have addressed the usefulness of polyamines in cancer chemotherapy. In more recent studies, polyamines have been identified as novel leads for the design of antidiarrhoeal agents and antimalarials and as ion chelators.

There is ever increasing realisation of the biological effects of polyamines, particularly in cellular processes, including growth and replication (Heby & Persson, Trends Bio. Sci., 1990, 15, 153). Thus, it is not surprising that polyamine conjugates continue to be the focus of significant attention as potential anticancer agents. It has been shown that a polyamine transporter specifically mediates the uptake of extracellular polyamines into cells (Seiler & Dezeure, Int. J. Biochem., 1990, 22, 211), and rapidly dividing tumour cells require large quantities of polyamines. Consequently, this polyamine transporter is up-regulated in tumour cells more so than in normal cells (Seiler et al., Cancer Res., 1990, 50, 5077). Polyamines bind to DNA via either the major or the minor groove (Rodger et al., Biopolymers, 1994, 34, 1583, Rogers et al., Bioorg. Med. Chem., 1995, 3, 861) and it is thought that endogenous polyamines also effect chromatin stability and structure (Basu et al., Biochem. J., 1992, 282, 723). Taking these aspects into account when designing polyamine based anticancer agents, there exists a potential uptake mechanism with selectivity for cancer cells (Cohen & Smith, Biochem. Soc. Trans., 1990, 18, 743) and two possible modes of cytotoxicity. This cytotoxicity may be mediated either by DNA binding and hence disruption of transcription (Feuerstein et al., Nucleic Acids Res., 1990, 18, 1271), or by interference with polyamine biosynthetic pathways thereby modulating the cellular concentrations of endogenous polyamines.

To date, some of the simplest and most effective synthetic polyamines to display anticancer activity have been developed by Porter, Bergeron and their co-workers. They initially found activity with spermidine and spermine analogues which are N-alkylated (Porter et al., Cancer Res., 1982, 42, 4072, Porter et al., Cancer Res., 1985, 45, 2050). Further studies showed the best analogues to be tetra-amines which have been bis-ethylated on the terminal, primary amines (e.g. compounds 9, 10 and 11) (Bergeron et al., J. Med. Chem., 1987, 31, 1183, Porter et al., Cancer Res., 1987, 47, 2821).

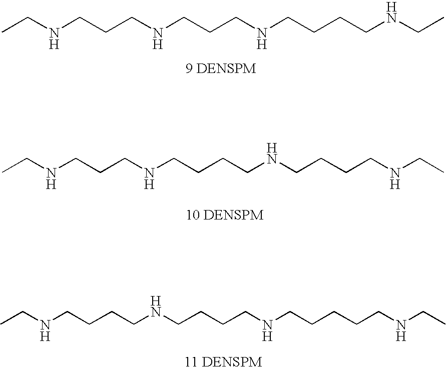

These compounds are recognised and taken into cells by the polyamine transporter. Once inside the cells, they deplete intracellular pools by down-regulating the enzyme ornithine decarboxylase (ODC), the first enzyme in the polyamine synthesis pathway, and up regulating the spermine-spermidine N1-acetyltransferase (SSAT) enzyme which works in the back conversion pathway (Bergeron et al., Cancer Res., 1989, 49, 2959). The cytotoxic effects of the analogues DENSPM (9), DESPM (10) and DEHSPM (11) in the in vitro culture of L1210 cells, over 96 h, were 1.3 μM, 0.2 μM and 0.06 μM respectively.

Many polyamine or polyamide moeities, for example desferrioxamine B (12), are low molecular weight ion chelating compounds. They facilitate iron solublization and transport.

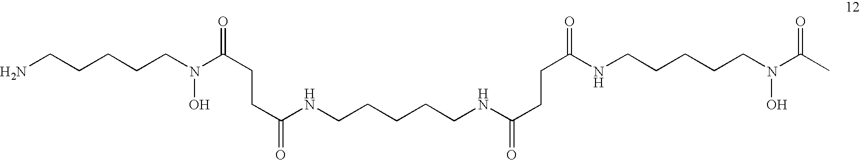

Another approach to the development of antitumour compounds is the covalent linking of cytotoxic agents, whose activity is mediated through direct interaction with DNA, to a polyamine. The resulting conjugate will be transported into the cell through the polyamine transport mechanism (if recognised) and the polyamine should further aid DNA binding of the cytotoxic component as its DNA target site. In line with this, Cullis has demonstrated that polyamines conjugated to the nitrogen mustard chlorambucil increase the efficiency of DNA alkylation at the N7 of guanine by factors in the range of 10³ to 10⁴ (Cullis et al., J. Am. Chem. Soc., 1995, 117, 8033).

It has now been discovered that artemisinin and synthetic trioxane derivatives can be chemically modified by the attachment of a polyamine residue to form analogues of artemisinin and synthetic trioxane derivatives which exhibit antimalarial, cytotoxic and antitumour activity.

According to a first aspect of the present invention therefore there is provided a compound of general formula 13 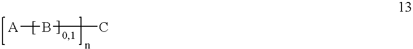
or a pharmaceutically acceptable salt thereof,
in which:
n=an integer of from 1 to 4;
A represents a trioxane-containing residue;
B represents a group having the general formula:
   -D-E-F-
in which:
   D is linked to A and represents an atom or group selected from the following: 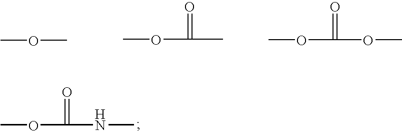
   E represents a bivalent, optionally substituted organic radical; and
   F is linked to C and represents a group selected from the following: 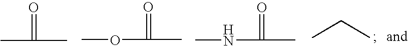
C represents a group containing at least two nitrogen atoms.

In a preferred embodiment of the present invention, A represents the following trioxane-containing residue:

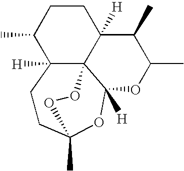

With regard to the optionally substituted organic radical E, this preferably comprises an organic radical of 2-50 carbon atoms, more preferably 1-20 carbon atoms. The optionally substituted organic radical E may comprise, for example, an optionally substituted alkyl, aryl, acyl, heteroalkyl or heteroacyl group. The organic radical E may optionally be substituted by groups including, but not limited to, primary, secondary and tertiary amines; halogen-containing groups, such as bromide, chloride and fluoride; alcohols and derivatives thereof, including ethers and esters; and carboxylic acids and derivatives thereof, including esters and amides. Examples of organic radicals comprising Group E include —CH₂—CH₂—, p-phenylene and pyridine.

With regard to group C, this may be, for example, a natural or synthetic polyamine residue and is preferably of 2-50 carbon atoms. Preferably also, group C comprises at least two amino groups, each of which is independently a primary or secondary group, after linking to group F through the same or different amino groups of the polyamine.

Those salts comprising pharmaceutically acceptable salts as referred to herein will be readily apparent to a skilled person. These salts include, but are not limited to acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, pamoate, phosphate, polygalacturonate, stearate, succinate, sulfate, sulfosalicylate, tannate, tartrate, terephthalate, tosylate, triethiodide, benzathine, calcium, diolamine, meglumine, olamine, potassium, procaine, sodium, tromethamine, and zinc.

Apart from imparting selectivity against cancer cells, the incorporation of amine functionality into an endoperoxide was seen as a useful strategy for enhancing antimalarial activity, since the amine peroxide should be concentrated in the acidic vacuole of the malaria parasite by ion-trapping (Vennerstrom et al., J. Med. Chem., 1989, 32, 64, O'Neill et al., J. Med. Chem., 1996, 39, 4511). Intraparasitic accumulation in the haem “rich” food vacuole is considered to be key to the action of all basic quinoline antimalarial agents (O'Neill, Pharm. & Therapeutics, 1998, 77, 29). For example, chloroquine is a dibasic drug with pKₐs of 8.1 (quinoline ring nitrogen) and 10.2 (diethylamino side chain) and accumulates in acidic vesicles to the square of the monobasic antimalarials such as mefloquine. Experiments on the pH gradient between the external medium and the parasite food vacuole have shown that the value is around 2.2. On this basis, Ginsburg and co-workers suggested that chloroquine would be expected to accumulate 2.5×10⁴ compared with 160 fold for the mono-basic antimalarials such as mefloquine (Ginsburg et al., Biochem. Pharmacol., 1989, 38, 2645). It follows that the introduction of two basic amino groups into an artemisinin derivative would be expected to increase significantly the cellular accumulation of drug in the ferrous rich parasite food vacuole. This approach should provide analogues with increased antimalarial potency, since more drug will be available for reductive endoperoxide bioactivation to radical species.

One preferred embodiment of the present invention provides compounds having the structure 13n. 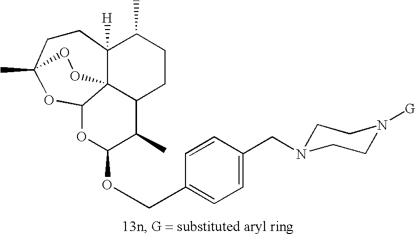
Specific examples of such compounds are included in Chart 1.

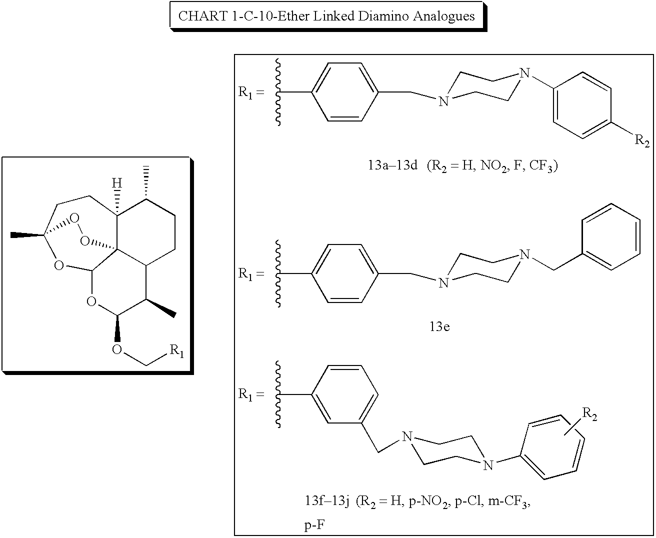

This series of preferred compounds was prepared as shown in Scheme 1. Dihydroartemisinin (2) was coupled with 1,4-benzenedimethanol to give the corresponding alcohol (12a) in high yield with excellent diastereoselectivity (β/α 5:1). The alcohol was then converted into the mesylate (12b) in high yield by treatment with mesyl chloride and triethylamine. The key mesylate was then allowed to react with a range of diamino nucleophiles to provide compounds (13a-13d). As shown in Scheme 1, DHA was also coupled with 1,3-benzenedimethanol to provide the alcohol which could then be transformed into target analogues (13f-13j) using the same chemistry described for the para substituted analogues. 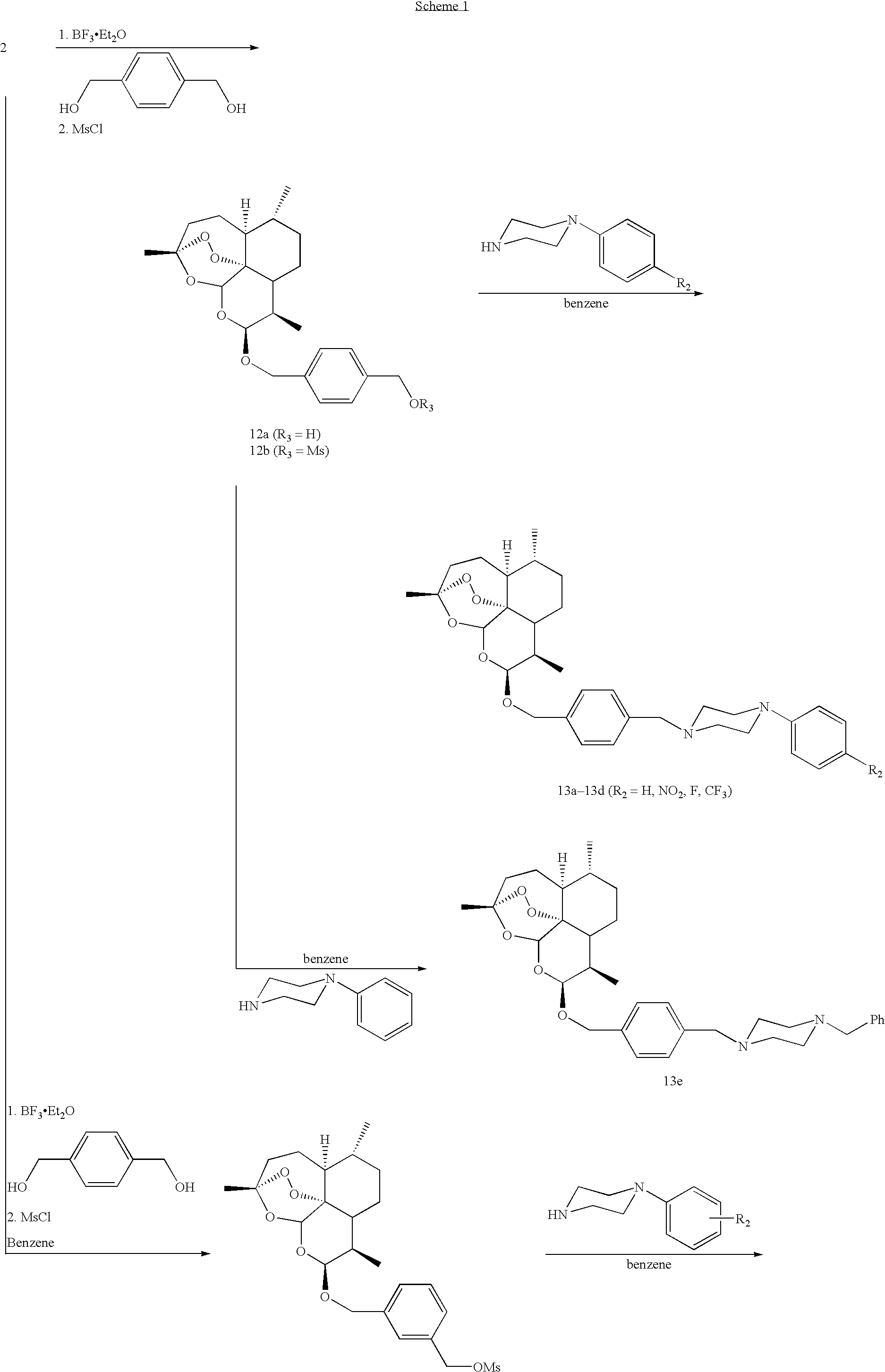 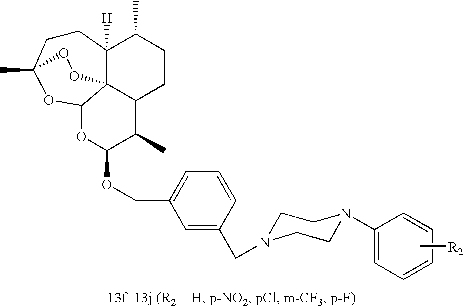
Thus, and in accordance with a further aspect of the present invention there is provided a process for the production of a compound of general formula 13 as hereinbefore defined, said process comprising the steps of coupling dihydroartemisinin with benzenedimethanol, converting the resultant alcohol into the corresponding sulfonate by treatment with a sulfonyl halide, and reacting said sulfonate with a diamino nucleophile.

Most preferably, the resultant alcohol is converted into the corresponding mesylate by treatment with mesyl chloride.

This process is particularly advantageous with regard to the production of trioxane derivatives of the type exemplified by compounds 13a-13j.

Further preferred compounds of the present invention were prepared according to Schemes 2 to 5 as shown below. Dihydroartemisinin (2) was coupled with a variety of alcohol methyl esters. These were then hydrolysed to give carboxylic acids, which were coupled with a number of polyamines and amines. Under standard conditions compounds with two artemisinin groups in the molecule were formed. If an excess of amine was used at 0° C., this gave the desired monomers.

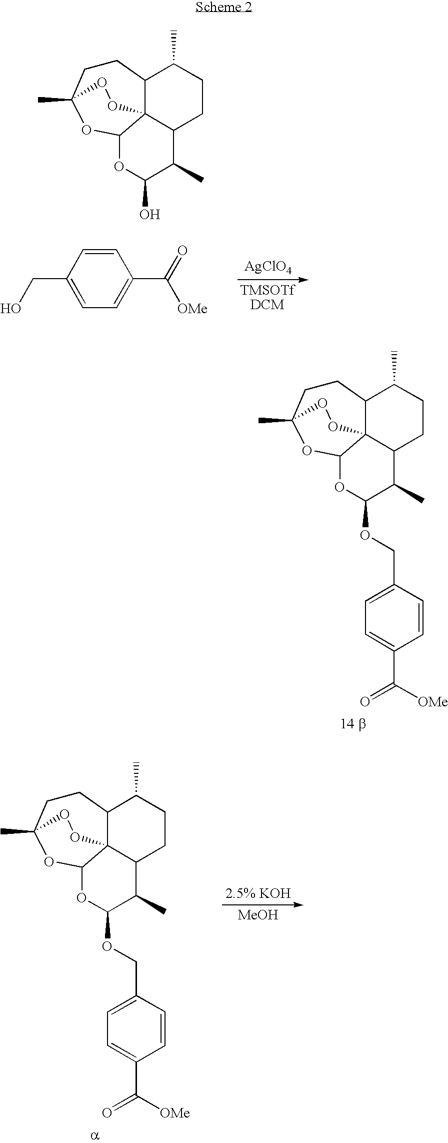

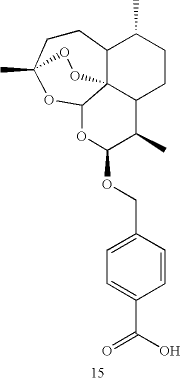

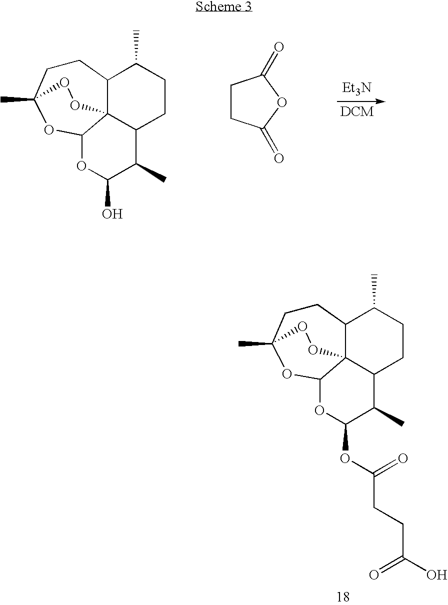

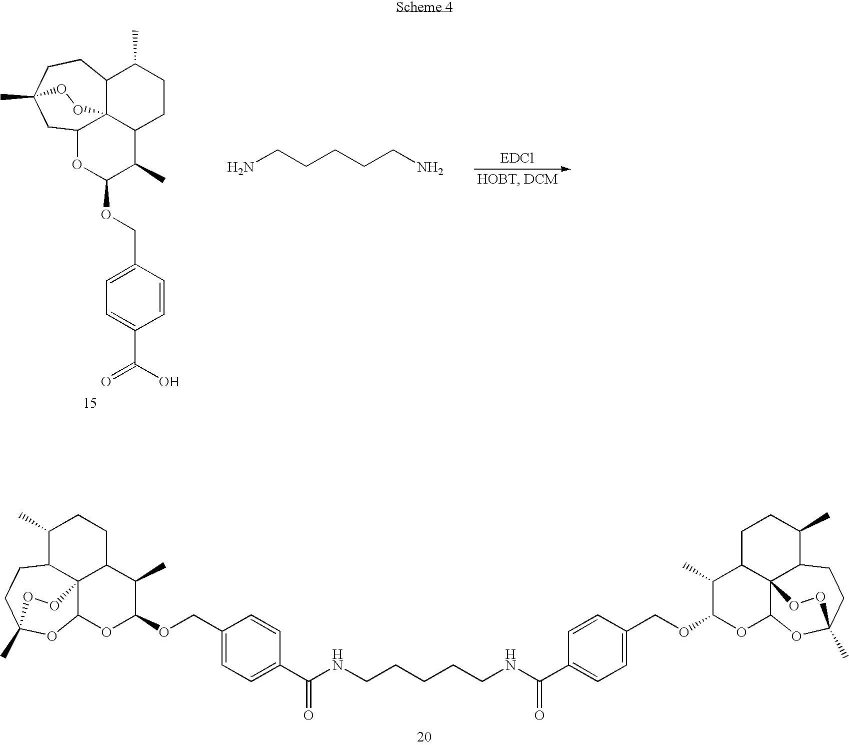

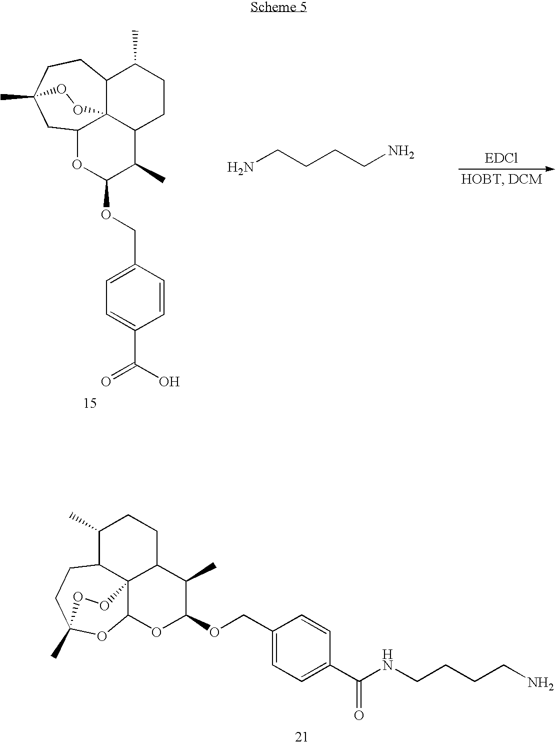

According to a further aspect of the present invention therefore there is provided a process for the production of a compound of general formula 13 as hereinbefore described, said process comprising the steps of coupling dihydroartemisinin with an alcohol methyl ester, hydrolysing the resultant compound to produce the corresponding carboxylic acid, and coupling said carboxylic acid with a polyamine or amine.

Alternatively, a further aspect of the invention provides a process for the production of a compound of general formula 13 as hereinbefore described, said process comprising the steps of coupling dihydroartemisinin with an anhydride, forming a carboxylic acid, and coupling said carboxylic acid with a polyamine or amine.

Preferably, coupling of the carboxylic acid and polyamine or amine is carried out at a temperature of between −20 and +40° C.

These processes are particularly advantageous with regard to the production of trioxane derivatives of the type exemplified by Examples 20 to 35.

The antimalarial activity of the new compounds was assessed using two strains of P. falciparum from Thailand: (a) the uncloned K1 strain which is known to be CQ resistant and (b) the HB3 strain which is sensitive to all antimalarials. Parasites were maintained in continuous culture using the method of Trager and Jenson (J. Parasitol., 1977, 63, 883-886). Cultures were grown in flasks containing human erythrocytes (2-5%) with parasitemia in the range of 1% to 10% suspended in RPMI 1640 medium supplemented with 25 mM HEPES and 32 mM NaHCO₃, and 10% human serum (complete medium). Cultures were gassed with a mixture of 3% O₂, 4% CO₂ and 93% N₂.

Antimalarial activity was assessed with an adaption of the 48-h sensitivity assay of Desjardins et al. (Antimicrob. Agents. Chemother., 1979, 16, 710-718) using [³H]-hypoxanthine incorporation as an assessment of parasite growth. Stock drug solutions were prepared in 100% dimethylsulphoxide (DMSO) and diluted to the appropriate concentration using complete medium. Assays were performed in sterile 96-well microtitre plates, each plate containing 200 μl of parasite culture (2% parasitemia, 0.5% haematocrit) with or without 10 μl drug dilutions. Each drug was tested in triplicate and parasite growth was compared to control wells (which constituted 100% parasite growth). After 24-h incubation at 37° C., 0.5 μCi hypoxanthine was added to each well. Cultures were incubated for a further 24 h before they were harvested onto filter-mats, dried for 1 h at 55° C. and counted using a Wallac 1450 Microbeta Trilux Liquid scintillation and luminescence counter. IC₅₀ values were calculated by interpolation of the probit transformation of the log dose—response curve.

The results of these experiments are contained in Table 1, which shows the IC50 of compounds of the present invention versus the K1 strain of P. falciparum in vitro.

TABLE 1

| Compound Number | IC50 (nM) | SD ± |
|---|---|---|
| (13a) | 8.2 | 2.7 |
| (13b) | 138.1 | 11.1 |
| (13c) | 6.3 | 0.7 |
| (13d) | 12.5 | 1.6 |
| (13e) | 3.8 | 0.1 |
| (13f) | 6.6 | 0.7 |
| (13g) | 4.3 | 0.30 |
| (13h) | 13.3 | 0.6 |
| (13i) | 22.5 | 1.9 |
| (13j) | 12.6 | 17 |
| Artemether | 6.5 | 2 |
| Chloroquine | 255 | 10 |

Table 2 shows the IC50 of compounds of the invention versus the HB3 strain of P. falciparum in vitro.

TABLE 2

| Compound Number | IC50 (nM) | SD ± |
|---|---|---|
| (13c) | 2.3 | 0.7 |
| (13d) | 8.1 | 1.2 |
| (13e) | 1.8 | 0.2 |
| (13f) | 3.4 | 0.7 |
| (13g) | 4.3 | 0.30 |
| (13j) | 12.6 | 17 |
| Artemether | 9.2 | 0.2 |

The anticancer activity of these compounds of the present invention was also assessed, by NCI 3-cell line anticancer assay. In this protocol, each cell line is inoculated and preincubated on a microtiter plate. Test agents are then added at a single concentration and the culture incubated for 48 hours. End-point determinations are made with sulforhodamine B, a protein-binding dye. Results for each test agent are reported as the percentage of growth of the treated cells when compared to the untreated control cells. Compounds which reduce the growth of any one of the cells lines to 32% or less (negative numbers indicate cell kill) are passed on for evaluation in the full panel of 60 cell lines over a 5-log dose range.

The results of these assays are shown in Table 3 below.

TABLE 3

| | | NCI-H460 | MCF7 | SF-268 |
|---|---|---|---|---|
| Compound Number | Concentration | (Lung) | (Breast) | (CNS) |
| (13h) | 1.00E−04Molar | 22 | 11 | 31 |
| (13i) | 1.00E−04Molar | 15 | 10 | 29 |
| (13c) | 1.00E−04Molar | 12 | 13 | 27 |

According to a further aspect of the present invention there is provided a compound of general formula 13 as hereinbefore defined, or a pharmaceutically acceptable salt thereof, for use as a medicament.

Compounds of the present invention may be used particularly, but not exclusively, as medicaments for the treatment of malaria or cancer. Whilst the currently preferred use of peroxides is for treatment, it cannot be ruled out that these compounds would have a use in the prophylaxis of malaria.

A therapeutically effective non-toxic amount of a compound of general formula 13 as hereinbefore defined may be administered in any suitable manner, including orally, parenterally (including subcutaneously, intramuscularly and intravenously), or topically. The administration will generally be carried out repetitively at intervals, for example once or several times a day.

The amount of the compound of general formula 13 that is required in order to be effective as an antimalarial or anticancer agent for treating human or animal subjects will of course vary and is ultimately at the discretion of the medical or veterinary practitioner treating the human or animal in each particular case. The factors to be considered by such a practitioner, e.g. a physician, include the route of administration and pharmaceutical formulation; the subject's body weight, surface area, age and general condition; and the chemical form of the compound to be administered.

In daily treatment, for example, the total daily dose may be given as a single dose, multiple doses, e.g. two to six times per day, or by intravenous infusion for any selected duration.

The compound of general formula 13 may be presented, for example, in the form of a tablet, capsule, liquid (e.g. syrup) or injection.

While it may be possible for the compounds of general formula 13 to be administered alone as the active pharmaceutical ingredient, it is preferable to present the compounds in a pharmaceutical composition.

According to a further aspect of the present invention therefore there is provided a pharmaceutical composition containing a compound of general formula 13 as hereinbefore defined, or a pharmaceutically acceptable salt thereof, as an active ingredient.

Such pharmaceutical compositions for medical use will be formulated in accordance with any of the methods well known in the art of pharmacy for administration in any convenient manner. The compounds of the invention will usually be admixed with at least one other ingredient providing a compatible pharmaceutically acceptable additive, carrier, diluent or excipient, and may be presented in unit dosage form.

The carrier(s) must be pharmaceutically acceptable in the sense of being compatible with the other ingredients of the formulation and not deleterious to the recipient thereof.

The possible formulations include those suitable for oral, rectal, topical and parenteral (including subcutaneous, intramuscular and intravenous) administration or for administration to the lung or another absorptive site such as the nasal passages.

All methods of formulation in making up such pharmaceutical compositions will generally include the step of bringing the compound of general formula 13 into association with a carrier which constitutes one or more accessory ingredients. Usually, the formulations are prepared by uniformly and intimately bringing the compound of general formula 13 into association with a liquid carrier or with a finely divided solid carrier or with both and then, if necessary, shaping the product into desired formulations.

Formulations of the present invention suitable for oral administration may be presented as discrete units such as capsules, cachets, tablets or lozenges, each containing a predetermined amount of the compound of general formula 13; as a powder or granules; or a suspension in an aqueous liquid or non-aqueous liquid such as a syrup, an elixir, an emulsion or a draught. The compound of general formula 13 may also be presented as a bolus, electuary or paste.

A tablet may be made by compression or moulding, optionally with one or more accessory ingredients. Compressed tablets may be prepared by compressing, in a suitable machine, the compound of general formula 13 in a free-flowing form such as a powder or granules, optionally mixed with a binder, lubricant, inert diluent, surface active or dispersing agent. Moulded tablets may be made by moulding, in a suitable machine, a mixture of the powdered compound of general formula 13 with any suitable carrier.

A syrup may be made by adding the compound of general formula 13 to a concentrated, aqueous solution of a sugar, for example sucrose, to which may be added any desired accessory ingredient. Such accessory ingredient(s) may include flavourings, an agent to retard crystallisation of the sugar or an agent to increase the solubility of any other ingredient, such as a polyhydric alcohol, for example glycerol or sorbitol.

Formulations for rectal administration may be presented as a suppository with a usual carrier such as cocoa butter.

Formulations suitable for parental administration conveniently comprise a sterile aqueous preparation of the compound of general formula 13 which is preferably isotonic with the blood of the recipient.

In addition to the aforementioned ingredients, formulations of this invention, for example ointments, creams and the like, may include one or more accessory ingredients, for example a diluent, buffer, flavouring agent, binder, surface active agent, thickener, lubricant and/or a preservative (including an antioxidant) or other pharmaceutically inert excipient.

The compounds of this invention may also be made up for administration in liposomal formulations which can be prepared by methods well-known in the art.

A further aspect of the present invention provides the use of a compound of general formula 13 as hereinbefore defined, or a pharmaceutically acceptable salt thereof, in the manufacture of a medicament for the treatment or prophylaxis of malaria.

A further aspect of the present invention provides the use of a compound of general formula 13 as hereinbefore defined, or a pharmaceutically acceptable salt thereof, in the manufacture of a medicament for the treatment of cancer.

It has further surprisingly been found that exposure of tumour cells to iron potentiates the anticancer effect of the compounds of the present invention. This finding is supported by the observation that cultured cancer cell lines were more readily destroyed by compounds of the present invention following exposure to culture medium having an elevated iron concentration.

According to a further aspect of the present invention therefore there is provided a product containing a first compound of general formula 13 as hereinbefore defined, or a pharmaceutically acceptable salt thereof, and a second, iron-containing, compound as a combined preparation for simultaneous, separate or sequential use in the treatment of cancers.

Preferably, the first and second compounds are used sequentially, the second, iron-containing, compound being used first.

The first compound may be presented in any of the forms described above. Administration of the first compound may be in any suitable manner, including intravenously, intraarterially, intralesionally, topically, intracavitarily or orally. Any suitable dosage of the compound may be used. Preferably, a dosage within the range of 0.1 to 500 mg/kg body weight is used, more preferably within the range of 0.5 to 300 mg/kg body weight, such as 1 to 50 mg/kg body weight.

With regard to the iron-containing compound, this may take any suitable form. Preferred agents for enhancing intracellular iron levels for use in the present invention include pharmaceutically acceptable iron salts and iron complexes. Iron salts useful in the present invention include ferrous fumarate, ferrous sulphate, ferrous carbonate, ferrous citrate, ferrous gluconate, ferrous lactate and ferrous maleate. Iron complexes useful in the present invention include ferrocholinate, ferroglycine sulphate, dextran iron complex, peptonized iron, iron sorbitex, saccharated iron, iron complexed with iron binding proteins and glycoproteins such as the holoferritins and holotransferrins.

The iron-containing compound may be presented in any of the forms described above in relation to the compound of general formula 13. Administration of the iron-containing compound may be achieved via any of the possible routes of administration of the first compound. The first and second compounds may be administered via the same or different routes.

The iron-containing compound may be used ay any appropriate dosage, but is preferably used at a dosage within the range of 0.01 to 1000 mg iron/kg body weight.

The product of the invention may further comprise one or more other agents known to be useful in the treatment of tumours. Such agents may include, for example, androgen inhibitors, antiestrogens, antimetabolites and cytotoxic agents.

According to a further aspect of the present invention there is provided a method of treatment of malaria which comprises administering to an animal in need of such treatment a therapeutically effective amount of a compound of general formula 13 as hereinbefore defined, or a pharmaceutically acceptable salt thereof.

In terms of suitable dosages for the treatment of malaria, the preferred amount of compounds of the present invention is between 10 mg to 5 g, preferably 50 to 1000 mg, administered over a period of 2-5 days, alone or in combination with other antimalarial drugs, such as, for example, the class II blood schizonticides or halofantrine (Looaeesuwan, Am. J. Trop. Med., 1999, 60, 238).

According to a further aspect of the present invention there is provided a method of treatment of cancer which comprises administering to an animal in need of such treatment a therapeutically effective amount of a compound of general formula 13 as hereinbefore defined, or a pharmaceutically acceptable salt thereof.

The method may further comprise the simultaneous, separate or sequential administration to the said animal an effective amount of an iron-containing compound as hereinbefore described.

The invention will now be illustrated by the following non-limiting examples.

### EXAMPLES

### Experimental Section

Merck Kieselgel 60 F 254 pre-coated silica plates for TLC were obtained from BDH, Poole, Dorset, U.K. Column chromatography was carried out on Merck 938S silica gel. Infra red (IR) spectra were recorded in the range 4000-600 cm⁻¹ using a Perkin Elmer 298 infrared spectrometer. Spectra of liquids were taken as films. Sodium chloride plates (nujol mull), solution cells (dichloromethane) and KBr discs were used as indicated.

¹H NMR spectra were recorded using a Perkin Elmer R34 (220 MHz) and Bruker (300 MHz and 200 MHz) spectrometers. Solvents are indicated in the text and tetramethylsilane was used as the internal reference. Mass spectra were recorded at 70 eV using a VG7070E mass spectrometer. The samples were introduced using a direct-insertion probe. In the text the parent ion (M⁺) is given, followed by peaks corresponding to major fragment losses with intensities in parentheses.

### General Procedure 1. Synthesis of Ether Derivatives

Dihydroartemisinin (2.00 g, 7.04 mmol) was dissolved in anhydrous diethyl ether (200 mL) under N₂. BF₃.Et₂O (1.03 mL, 8.10 mmol) was added to the solution, followed by the appropriate (hydroxymethyl)benzyl alcohol (1.46 g, 10.56 mmol). The mixture was allowed to stir at room temperature for 20 h and then quenched with water. The organic phase was washed with Na₂SO₄ solution (30% w/v), dried over MgSO₄, filtered and the solvent was removed under reduced pressure to the give the crude product as an oil. Purification by silica gel chromatography using ethyl acetate/ⁿhexane (40/60) as the eluent gave the corresponding ether products.

### 10β-[[4-(Hydroxymethyl)benzyl]oxy]dihydroartemisinin (12a)

This compound was prepared using general procedure 1 to give the product as a colourless syrup (78% yield): ¹H NMR (300 MHz, CDCl₃) δ 7.20-7.08 (4H, m, aromatic), 5.44 (1H, s), 4.88 (1H, d, J=3.80 Hz), 4.85 (1H, d, J=12.19 Hz), 4.69 (2 H, s), 4.51 (1H, d, J=12.19 Hz), 2.67 (1H, sex), 2.38 (1H, dt, J=13.46, 3.98 Hz), 2.07-1.20 (10H, m), 1.46 (3H, s) and 0.94 (6H, d, J=7.17 Hz); ¹³C NMR (75 MHz, CDCl₃) δ 140.20, 137.88, 127.59, 127.05, 104.21, 101.45, 88.09, 81.18, 69.57, 65.11, 52.62, 44.45, 37.43, 36.46, 34.64, 30.94, 26.16, 24.69, 24.52, 20.32 and 13.06; IR (thin film)/cm⁻¹ 3476, 2924, 1612, 1516, 1458, 1377, 1194, 1101, 1011, 876 (O-O) and 826 (O-O); MS m/z (CI) [M+NH₄]⁺ 422 (8), 359 (100), 284 (39), 221 (96) and 138 (33).

### 10β-[[3-(Hydroxymethyl)benzyl]oxy]dihydroartemisnin

This compound was prepared using general procedure 1 to give the product as a colourless solid (68% yield): m.p. 118-120° C.; ¹H NMR (300 MHz, CDCl₃) δ 7.34-7.26 (4H, m, aromatic), 5.40 (1H, s), 4.92 (1H, d, J=3.80 Hz), 4.88 (1H, d, J=12.20 Hz), 4.70 (2H, s), 4.55 (1H, d, J=12.20 Hz), 2.67 (1H, sex), 2.38 (1H, dt, J=13.50, 3.80 Hz), 2.07-1.20 (10H, m), 1.45 (3H, s), 0.96 (3H, d, J=6.00 Hz) and 0.88 (3H, d, J=7.60 Hz); ¹³C NMR (75 MHz, CDCl₃) δ 141.05, 138.93, 128.64, 126.80, 126.15, 104.21, 101.65, 88.06, 81.17, 69.90, 65.37, 52.62, 44.47, 37.43, 36.46, 34.65, 30.97, 26.16, 24.70, 24.51, 20.31 and 13.07; IR (Nujol)/cm⁻¹ 3507, 2924, 1611, 1462, 1378, 1227, 1192, 1104, 1011, 874 (O-O) and 823 (O-O); Anal. C₂₃H₃₂O₆ requires C 68.29%, H 7.97%, found C 68.01%, H 8.14%.

### General Procedure 2. Synthesis of C-10 Oxo Derivatives

To a solution of the appropriate (hydroxymethyl)benzyl alcohol (0.20 g, 0.50 mmol) in anhydrous DCM (10 mL) under N₂ was added triethylamine (0.08 mL, 0.55 mmol), followed by mesyl chloride (0.06 mL, 0.74 mmol) at 0° C. The mixture was stirred at 0° C. for 2 h and then quenched with water (10 mL). The organic phase was extracted with DCM (3×10 mL) and then dried over MgSO₄, filtered and the solvent was removed under reduced pressure. The crude mesylate and the appropriate substituted piperazine derivative (4.11 mmol) were dissolved in anhydrous benzene (10 mL) under N₂ atmosphere. The mixture was heated at reflux for 5 h. After allowing to cool to room temperature, the mixture was quenched with saturated NaHCO₃ solution and the organic phase was extracted with diethyl ether (3×10 mL). The organic extracts were washed with brine, dried over MgSO₄, filtered and the solvent was removed under reduced pressure. Purification by silica gel chromatography using ethyl acetate/ⁿhexane (40/60) as the eluent gave the corresponding piperazine products.

### 10β-[[4-[Phenylpiperazyl)methyl]benzyl]dihydroartemisinin (13a)

This compound was prepared from 1-phenyl piperazine using general procedure 2 to give the product as a yellow oil (84% yield): ¹H (300 MHz, CDCl₃) δ 7.30-7.28 (4H, d, J=4.5 Hz, aromatic), 7.00-6.90 (5H, m, aromatic), 5.45 (1H, s), 4.90 (1H, d, J=3.80 Hz), 4.86 (1H, d, J=12.20 Hz), 4.50 (1H, d, J=12.20 Hz), 3.53 (2H, s, CH₂), 2.69 (1H, m, CH), 2.51 (8H, m, CH₂), 2.38 (1H, dt, J=13.32, 4.12 Hz), 2.07-1.20 (10H, m), 1.45 (3 H, s, CH₃), 0.95-0.93 (6H, 2×CH₃); C (75 MHz, CDCl₃) 129.34, 128.28, 127.19, 104.16, 101.51, 88.08, 81.18, 69.67, 62.98, 62.68, 52.92, 52.66, 44.49, 37.43, 36.49, 34.67, 30.97, 26.19, 24.71, 24.52, 20.31, 13.06; LC/MS (NH₃); m/z 563[M+H⁺, (100)], 279 (17).

### 10β-[[4-[(4-Nitrophenylpiperazyl)methyl]]benzyl]oxy]dihydroartemisinin (13b)

This compound was prepared from 1-(4-nitrophenyl)piperazine using general procedure 2 to give the product as an orange solid (68% yield): ¹H (300 MHz, CDCl₃) δ 8.12 (2H, d, J=9.48 Hz, aromatic), 7.35-7.27 (4H, m, aromatic), 6.82 (2H, d, J=9.48 Hz, aromatic), 5.47 (1H, s), 4.91 (1H, d, J=3.90 Hz), 4.90 (1H, d, J=12.50 Hz), 4.53 (1 H, d, J=12.50 Hz), 3.58 (2H, s), 3.47-3.42 (4H, m), 2.68 (1H, m), 2.64-2.59 (4H, m), 2.38 (1H, dt, J=13.32, 4.12 Hz), 2.07-1.20 (10H, m), 1.46 (3H, s) and 0.95 (6H, d, J=6.70 Hz); ¹³C (75 MHz, CDCl₃) δ 154.96, 129.17, 127.34, 125.99, 112.70, 104.19, 101.43, 88.09, 81.16, 69.54, 62.61, 52.63, 52.48, 47.08, 44.46, 37.46, 36.47, 34.67, 30.94, 26.18, 24.71, 24.53, 20.31 and 13.06; IR (Nujol)/cm⁻¹ 2927, 1597, 1493, 1462, 1377, 1327, 1251, 1231, 1099, 1010, 876 (O-O) and 828 (O-O); MS m/z (EI) [M]⁺ 593 (1), 264 (98), 218 (34), 104 (100) and 56 (58).

### 10β-[[4-[(4-Fluorophenylpiperazyl)methyl]benzyl]oxy]dihydroartemisnin (13c)

This compound was prepared from 1-(4-fluorophenyl)piperazine using general procedure 2 to give the product as a yellow oil (64% yield): ¹H (300 MHz, CDCl₃) δ 7.35-7.28 (4 H, m, aromatic), 6.99-6.85 (4H, m, aromatic), 5.46 (1H, s), 4.91 (1H, d, J=3.90 Hz), 4.90 (1H, d, J=12.45 Hz), 4.52 (1H, d, J=12.45 Hz), 3.58 (2H, s), 3.16-3.11 (4H, m), 2.68-2.63 (5H, m), 2.38 (1H, dt, J=13.50, 3.90 Hz), 2.07-1.20 (10H, m), 1.46 (3H, s), 0.95 (3H, d, J=6.00 Hz) and 0.94 (3H, d, J=6.60 Hz); ¹³C (75 MHz, CDCl₃) δ 141.05, 138.93, 128.64, 126.80, 126.15, 104.21, 101.65, 88.06, 81.17, 69.90, 65.37, 52.62, 44.47, 37.43, 36.46, 34.65, 30.97, 26.16, 24.70, 24.51, 20.10 and 13.07; IR (thin film)/cm⁻¹ 2945, 1633, 1510, 1455, 1374, 1359, 1240, 1142, 1099, 1011, 876 (O-O) and 825 (O-O); HRMS (EI) C₃₃H₄₃FN₂O₅ [M]⁺ requires 566.31561, found 566.31493.

### 10β-[[4-[(4-Trifluoromethylphenylpiperazyl)methyl]benzyl]oxy]dihydroartemisinin (13d)

This compound was prepared from 1-(4-trifluoromethyl phenyl)piperazine using general procedure 2 to give the product as a yellow oil (64% yield): ¹H (300 MHz, CDCl₃) δ 7.35-7.28 (5H, m, aromatic), 7.10-7.03 (3H, m, aromatic), 5.46 (1H, s), 4.92 (1H, d, J=12.30 Hz), 4.88 (1H, d, J=3.80 Hz), 4.52, (1H, d, J=12.30 Hz), 3.61 (2H, m, CH₂), 3.27 (4H, m, CH₂), 2.70-2.65 (5H, m, CH₂), 2.39 (1H, m, CH₂), 2.07-1.20 (10H, m), 1.45 (3H, s, CH₃), 0.97-0.94 (6H, d, 2×CH₃); ¹³C (75 MHz, CDCl₃) δ 129.59, 129.32, 127.33, 118.78, 112.29, 104.19, 101.50, 88.09, 81.17, 69.61, 62.58, 52.73, 48.55, 44.48, 37.46, 36.48, 34.67, 30.96, 26.18, 24.71, 24.54, 20.31, 13.06; IR (thin film)/cm⁻¹ (2925), (1454), (1136), (1011). LC/MS (NH₃); m/z 618 [M+H⁺, (100)], 603 (100), 333 (7).

### 10β-[[4-[(Benzylpiperazyl)methyl]benzyl]oxy]dihydroartemisinin (13c)

This compound was prepared from 1-benzylpiperazine using general procedure 2 to give the product as a yellow oil (72% yield): ¹H NMR (300 MHz, CDCl₃) δ 7.32-7.20 (9H, m, aromatic), 5.45 (1H, s), 4.91 (1H, d, J=3.90 Hz), 4.90 (1H, d, J=12.50 Hz), 4.52 (1H, d, J=12.50 Hz), 3.54 (4H, br s), 2.68 (1H, m), 2.54-2.49 (8H, m), 2.38 (1H, dt, J=14.10, 3.90 Hz), 2.07-1.20 (10H, m), 1.45 (3H, s), 0.94 (3H, d, J=5.70 Hz) and 0.94 (3H, d, J=7.2 Hz); ¹³C (75 MHz, CDCl₃) δ 129.34, 128.28, 127.19, 104.16, 101.51, 88.08, 81.18, 69.67, 62.98, 62.68, 52.92, 52.66, 44.49, 37.43, 36.49, 34.67, 30.97, 26.19, 24.71, 24.52, 20.31 and 13.06; IR (thin film)/cm⁻¹ 2938, 1609, 1495, 1457, 1374, 1344, 1227, 1099, 1010, 876 (O-O) and 826 (O-O).

### 10β-[[3-[(Phenylpiperazyl)methyl]benzyl]oxy]dihydroartemisinin (13f)

This compound was prepared from 1-phenylpiperazine using general procedure 2 to give the product as a brown foam (59% yield): ¹H NMR (300 MHz, CDCl₃) δ 7.30-7.22 (4H, m, aromatic), 6.94-6.85 (5H, m, aromatic), 5.47 (1H, s), 4.91 (1H, d, J=3.70 Hz), 4.90 (1H, d, J=12.20 Hz), 4.55 (1H, d, J=12.20 Hz), 3.59 (2H, br s), 3.27-3.21 (4H, m), 2.69-2.61 (5H, m), 2.38 (1H, dt, J=13.30, 3.80 Hz), 2.07-1.20 (10H, m), 1.46 (3H, s), 0.95 (3H, d, J=7.40 Hz) and 0.94 (3H, d, J=6.00 Hz); ¹³C (75 MHz, CDCl₃) δ 139.10, 129.17, 128.38, 116.16, 104.19, 101.40, 88.10, 81.18, 69.68, 52.65, 44.47, 37.45, 36.48, 34.69, 30.96, 26.19, 24.71, 24.53, 20.32 and 13.10; IR (Nujol)/cm⁻¹ 2925, 1601, 1504, 1455, 1375, 1228, 1101, 1013, 875 (O-O) and 825 (O-O); HRMS (EI) C₃₃H₄₄N₂O₅ [M]⁺ requires 548.32501, found 548.32604.

### 10β-[[3-[(4-Nitrophenylpiperazyl)methyl]benzyl]oxy]dihydroartemisinin (13g)

This compound was prepared from 1-(4-nitrophenyl)piperazine using general procedure 2 to give the product as an orange foam (85% yield): ¹H (300 MHz, CDCl₃) δ 8.13 (2H, d, J=9.50 Hz, aromatic), 7.36-7.25 (4H, m, aromatic), 6.82 (2H, d, J=9.50 Hz, aromatic), 5.47 (1H, s), 4.91 (1H, d, J=5.00 Hz), 4.90 (1H, d, J=12.30 Hz), 4.56 (1 H, d, J=12.30 Hz), 3.61 (2H, s), 3.49-3.43 (4H, m), 2.71 (1H, m), 2.69-2.63 (4H, m), 2.39 (1H, dt, J=13.87, 3.98 Hz), 2.07-1.20 (10H, m), 1.45 (3H, s) and 0.94 (6H, d, J=7.40 Hz); ¹³C (75 MHz, CDCl₃) δ 151.00, 128.50, 128.00, 125.99, 112.79, 104.00, 101.00, 88.09, 81.50, 69.56, 60.38, 52.61, 52.20, 46.96, 44.20, 37.47, 36.45, 34.50, 30.93, 26.00, 24.70, 24.55, 20.32 and 13.00; IR (Nujol)/cm⁻¹ 2923, 1598, 1506, 1456, 1378, 1328, 1248, 1099, 1010, 875 (O-O) and 826 (O-O); MS m/z (EI) [M]⁺ 593 (1), 264 (43), 219 (18), 105 (100) and 56 (30).

### 10β-[[3-[(4-Chlorophenylpiperazyl)methyl]benzyl]oxy]dihydroartemisinin (13h)

This compound was prepared from 1-(4-chlorophenyl)piperazine using general procedure 2 to give the product as a brown foam (64% yield): ¹H (300 MHz, CDCl₃) δ 7.32-7.06 (6 H, m, aromatic), 6.83 (2H, d, J=9.06, aromatic), 5.47 (1H, s), 4.92 (1H, d, J=4.10 Hz), 4.90 (1H, d, J=12.01 Hz), 4.55 (1H, d, J=12.01 Hz), 3.61 (2H, br s), 3.22-3.17 (4H, m), 2.69-2.61 (5H, m), 2.39 (1H, dt, J=13.50, 3.85 Hz), 2.07-1.20 (10H, m), 1.46 (3H, s) and 0.94 (3H, d, J=6.60 Hz); ¹³C (75 MHz, CDCl₃) δ 138.50, 129.03, 128.44, 117.35, 104.19, 101.37, 88.09, 81.17, 69.64, 52.81, 49.03, 44.45, 37.45, 36.47, 34.68, 30.94, 26.19, 24.71, 24.53, 20.32 and 13.10; IR (Nujol)/cm⁻¹ 2927, 1616, 1496, 1459, 1378, 1225, 1102, 1032, 874 (O-O) and 815 (O-O); MS m/z (EI) [M]⁺ 422 (1), 300 (26), 193 (19), 131 (14) and 105 (100).

### 10β-[[3-[(3-Trifluoromethylphenylpiperazyl)methyl]benzyl]oxy]dihydroartemisinin (13i)

This compound was prepared from 1-[(3-trifluoromethyl)phenyl]piperazine using general procedure 2 to give the product as a brown foam (66% yield): ¹H (300 MHz, CDCl₃) δ 7.37-7.23 (4H, m, aromatic), 7.10-7.06 (4H, m, aromatic), 5.47 (1H, s), 4.92 (1H, d, J=3.98 Hz), 4.90 (1H, d, J=12.16 Hz), 4.52 (1H, d, J=12.16 Hz), 3.58 (2H, s), 3.16-3.11 (4H, m), 2.68-2.63 (5H, m), 2.38 (1H, dt, J=13.48, 4.02 Hz), 2.07-1.20 (10H, m), 1.45 (3H, s), 0.95 (3H, d, J=7.20 Hz) and 0.94 (3H, d, J=6.00 Hz); ¹³C NMR (75 MHz, CDCl₃) δ 151.49, 138.65, 129.59, 128.40, 118.74, 115.81, 112.22, 104.19, 101.38, 88.09, 81.17, 69.68, 62.81, 52.80, 52.64, 48.64, 44.46, 37.46, 36.46, 34.69, 30.95, 26.19, 24.71, 24.53, 20.31 and 13.10; IR (Nujol)/cm⁻¹ 2921, 1612, 1496, 1454, 1350, 1228, 1120, 1011, 875 (O-O) and 826 (O-O); MS m/z (EI) [M]⁺ 616 (1), 334 (11), 227 (7), 105 (100) and 56 (10).

### 10β-[[3-[(4-Fluorophenylpiperazyl)methyl]benzyl]oxy]dihydroartemisinin (13j)

This compound was prepared from 1-(4-fluorophenyl)piperazine using general procedure 2 to give the product as an off-white foam (69% yield): ¹H (300 MHz, CDCl₃) δ 7.36-7.27 (4H, m, aromatic), 6.98-6.84 (4H, m, aromatic), 5.47 (1H, s), 4.91 (1H, d, J=3.43 Hz), 4.90 (1H, d, J=12.29 Hz), 4.54 (1H, d, J=12.29 Hz), 3.58 (2H, s), 3.12 (4H, t, J=4.81 Hz), 2.63 (1H, m), 2.62 (4H, t, J=13.80 Hz), 2.38 (1H, dt, J=13.40, 3.90 Hz), 2.07-1.20 (10H, m), 1.45 (3H, s), 0.94 (3H, d, J=6.00 Hz) and 0.93 (3H, d, J=6.00 Hz); ¹³C (75 MHz, CDCl₃) δ 148.04, 138.62, 128.40, 126.34, 117.89, 115.70, 115.40, 104.19, 101.36, 88.09, 81.18, 69.65, 62.82, 52.99, 52.64, 50.07, 44.46, 37.45, 36.47, 34.69, 30.95, 26.19, 24.71, 24.53, 20.32 and 13.10; IR (Nujol)/cm⁻¹ 2924, 1510, 1460, 1377, 1229, 1160, 1102, 1012, 875 (O-O) and 826 (O-O); HRMS (EI) C₃₃H₄₃FN₂O₅ [M]+requires 566.31561, found 566.31437; C₃₃H₄₃FN₂O₅ requires C 69.97%, H 7.60%, N 4.97%, found C 69.67%, H 7.72%, N 4.82%.

### Procedure 1. Synthesis of β methyl-p-[(10-dihydroartemisininoxy)methyl]benzoate (14)

A solution of dihydroartemisinin (5 g, 17.6 mmol) in anhydrous dichloromethane (40 ml) was stirred at room temperature. Methyl 4-(hydroxymethyl)-benzoate (6.7 g, 40 mmol) and silver perchlorate (0.73 g, 3.52 mmol) were added. The reaction was cooled to −78° C. and stirred under N₂. Trimethylsilyl triflate was added dropwise and the reaction mixture was stirred under N₂ at −78° C. for 2 hours. The reaction was quenched with triethylamine (20 ml) and allowed to warm to room temperature. Any resulting precipitate was removed by filtration and the reaction mixture was evaporated to dryness. Purification of the crude mixture by flash column chromatography, using 10% ethyl acetate/n-hexane as the eluent, gave the β (14) and α-isomers in 47% and 14% yield respectively.

Data for β-isomer 14 ¹H NMR (250 MHz, CDCl₃) 7.95 (2H, d, J=8.3 Hz, aromatic), 7.31 (2H, d, J=8.3 Hz, aromatic), 5.38 (1H, s), 4.89 (1H, d, J=13.2 Hz, AB coupling), 4.85 (1H, d, J=3.7 Hz), 4.50 (1H, d, J=13.2 Hz, AB coupling), 3.84 (3H, s, OMe), 2.62 (1H, m, CH), 2.28 (1H, dt, J=13.4, 4.0 Hz), 2.00-1.16 (10H, m), 1.45 (3H, s, CH₃), 0.91-0.86 (6H, 2×CH₃).

### Procedure 2. Synthesis of β-artelinic Acid (15)

A suspension of 14β (3.65 g, 8,42 mmol) in aqueous potassium hydroxide and methanol (2.5% KOH/MeOH, 1/1 mixture, 350 ml) was stirred for 24 hours at room temperature. After this time the methanol was removed in vacuo and the aqueous mixture was cooled to 0° C. The mixture was acidified to pH 2 by dropwise addition of dilute hydrochloric acid, then extracted with diethyl ether (3×200 ml). The organic extracts were dried over anhydrous MgSO₄ and the solution was evaporated to dryness. This gave pure product 2 as a white foam in a 92% yield: ¹H NMR (250 MHz, CDCl₃) 8.90 (2H, d, J=8.3 Hz, aromatic), 7.44 (2H, d, J=8.3 Hz, aromatic), 5.47 (1H, s), 5.00 (1H, d, J=13.3 Hz, AB coupling), 4.90 (1H, d, J=3.4 Hz), 4.62 (1H, d, J=13.3 Hz, AB coupling), 2.72 (1H, m, CH), 2.38 (1H, dt, J=13.4, 4.0 Hz), 2.08-1.20 (11H, m), 1.47 (3H, s, CH₃), 1.01-0.95 (6H, 2×CH₃).

### Synthesis of Example 16

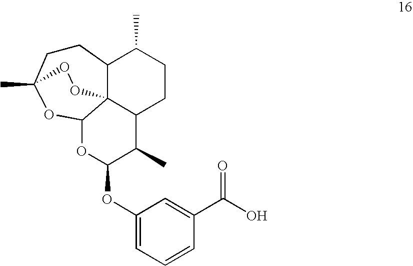

This compound was prepared from methyl 3-hydroxybenzoate using procedure 1 and procedure 2 to give the product as a yellow foam (77% yield): ¹H NMR (250 MHz, CDCl₃) 7.80 (1H, s, aromatic), 7.75 (1H, m, aromatic), 7.43-7.40 (2H, 2×d, J=5.4 Hz, aromatic), 5.59 (1H, d, J=2.8 Hz), 5.51 (1H, s), 2.86 (1H, m, CH), 2.40 (1H, dt, J=13.2, 3.9 Hz), 2.08-1.20 (11H, m), 1.47 (3H, s, CH₃), 1.07-0.98 (6H, 2×CH₃).

### Synthesis of Example 17

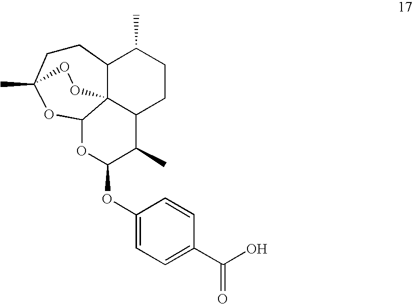

This compound was prepared from methyl 4-hydroxybenzoate using procedure 1 and procedure 2 to give the product, as a yellow foam (45% yield).

Hydrolysis of the ester was carried out by the same procedure as for the synthesis of 15 but using a solvent mix of 2.5% aqueous KOH, MeOH and THF in a 2/1/1 ratio: ¹H NMR (250 MHz, CDCl₃) 8.07 (2H, d, J=8.7 Hz, aromatic), 7.18 (2H, d, J=8.7 Hz, aromatic), 5.62 (1H, d, J=3.2 Hz), 5.46 (1H, s), 2.85 (1H, m, CH), 2.39 (1H, dt, J=14.2, 4.0 Hz), 2.13-1.20 (11H, m), 1.46 (3H, s, CH₃), 1.05-0.95 (6H, 2×CH₃).

### Procedure 3. Synthesis of 10 Succinyl Dihydroartemisinin (18)

A solution of dihydroartemisinin (1.5 g, 5.28 mmol) in anhydrous dichloromethane (75 ml) was stirred at room temperature. Succinic anhydride (0.63 g, 6.33 mmol) and triethyl amine (3.7 ml, 26.4 mmol) were added and the solution was stirred at room temperature for 1 hour. The reaction mixture was washed with aqueous citric acid (pH 2, 2×20 ml), dried over MgSO₄ and evaporated to dryness. Purification of the crude mixture by flash column chromatography, using 2-5% methanol/dichloromethane as the eluent, gave 18 in a 65% yield: ¹H NMR (250 MHz, CDCl₃) 5.73 (1H, d, J=9.9 Hz), 5.36 (1H, s), 2.62 (2H, m), 2.64 (4H, dd, J=4.4, 1.9 Hz), 2.30 (1H, dt, J=13.3, 4.0 Hz), 1.99-1.18 (10H, m), 1.36 (3H, s, CH₃), 0.89 (3H, d, J=5.8 Hz, CH₃), 0.78 (3H, d, J=7.1 Hz, CH₃).

### Synthesis of Example 19

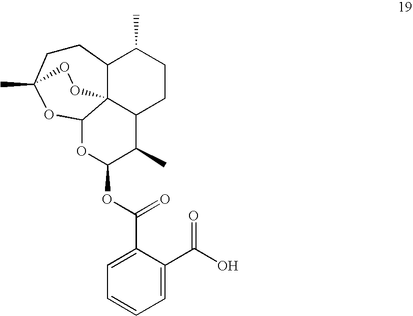

This compound was prepared from phthalic anhydride using procedure 3 to give 19 in a 70% yield: ¹H NMR (250 MHz, CDCl₃) 7.78 (2H, m, aromatic), 7.52 (2H, m, aromatic), 5.91 (1H, d, J=9.8), 5.56 (1H, s), 2.58 (1H, m, CH), 2.30 (1H, dt, J=14.1, 3.9 Hz), 2.00-1.20 (11H, m), 1.36 (3H, s, CH₃), 0.91-0.84 (6H, 2×CH₃).

### Procedure 4. Synthesis of Example 20

A solution of 15 (100 mg, 0.24 mmol), 1-(3-dimethylaminopropyl)-3-ethylcarbodiimide hydrochloride (46 mg, 0.24 mmol) and 1-hydroxybenzotriazole (32 mg, 0.24 mmol) in dichloromethane (2 ml) was stirred at room temperature. 1,5-diaminopentane (0.14 ul, 0.12 mmol) was added and the reaction was stirred for 2 hours at room temperature. After this time the reaction was washed with water (3×20 ml), the organic extracts dried over MgSO₄ and evaporated to dryness. Purification of the crude mixture, using preparative HPLC (C18, Luna, 100×21.2 mm, 10 micron), gave 20 in a 14% yield: ¹H NMR (250 MHz, CDCl₃) 7.68 (4H, d, J=8.2 Hz, aromatic), 7.28 (4H, d, J=8.2 Hz, aromatic), 6.31 (2H, m, amide) 5.37 (2H, s), 4.89 (2H, d, J=12.9 Hz, AB coupling), 4.83 (2H, s), 4.49 (2H, d, J=12.9 Hz, AB coupling), 3.40 (4H, m) 2.61 (2H, m, CH), 2.26 (2H, dt, J=13.5, 4.0 Hz), 2.08-1.16 (26H, m), 1.38 (6H, s, CH₃), 0.89-0.83 (12H, 2×CH₃).

### Procedure 5. Synthesis of Example 21

A solution of 1,4-diaminobutane (0.17 g, 1.9 mmol) in dichloromethane (6 ml) was cooled to 0° C. A pre-formed solution of 15 (200 mgs, 0.47 mmol), 1-(3-dimethylaminopropyl)-3-ethylcarbodiimide hydrochloride (92 mg, 0.47 mmol) and 1-hydroxybenzotriazole (65 mg, 0.47 mmol) in dichloromethane (2 ml) was added dropwise over 1 hour. The reaction mixture was stirred at 0° C. for a further hour, washed with water (3×20 ml), the organic extracts dried over MgSO₄ and evaporated to dryness. Purification of the crude mixture by flash column chromatography, using 50% methanol/dichloromethane as the eluent, gave product 21 in a 21% yield: ¹H NMR (250 MHz, CDCl₃) 7.70 (2H, d, J=8.2 Hz, aromatic), 7.28 (2H, d, J=8.2 Hz, aromatic), 7.00 (1H, m, amide) 5.37 (1H, s), 4.85 (1H, d, J=12.7 Hz, AB coupling), 4.8 (1H, s), 4.48 (1H, d, J=12.7 Hz, AB coupling), 3.38 (2H, m) 2.60 (2H, m, CH, NH), 2.26 (1H, dt, J=14.3, 4.0 Hz), 2.01-1.15 (17H, m), 1.38 (3H, s, CH₃), 0.89-0.83 (6H, 2×CH₃).

### Synthesis of Example 22

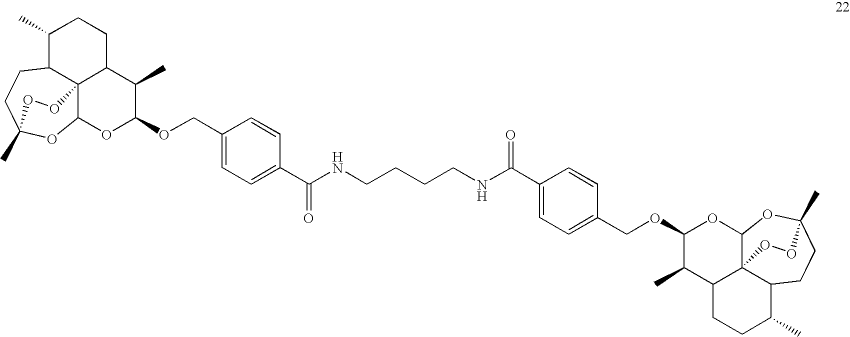

This compound was prepared from compound 15 and 1,4-diaminobutane, using a similar procedure to 4. Purification of the crude material by preparative HPLC gave the product in a 31% yield: ¹H NMR (250 MHz, CDCl₃) 7.80 (4H, d, J=8.2 Hz, aromatic), 7.39 (4H, d, J=8.2 Hz, aromatic), 6.61 (2H, m, amide) 5.46 (2H, s), 4.95 (2H, d, J=12.7 Hz, AB coupling), 4.91 (2H, s), 4.57 (2H, d, J=12.7 Hz, AB coupling), 3.52 (4H, m) 2.69 (2H, m, CH), 2.34 (2H, dt, J=13.6, 3.9 Hz), 2.10-1.24 (24H, m), 1.46 (6H, s, CH₃), 0.98-0.94 (12H, 2×CH₃).

### Synthesis of Example 23

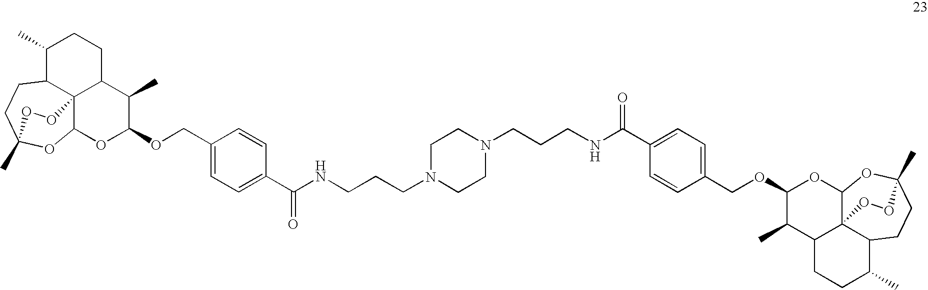

This compound was prepared from compound 15 and 1,4-bis(3-aminopropyl)-piperazine, using a similar procedure to 4 to give the product in a 21% yield: ¹H NMR (250 MHz, CDCl₃) 8.12 (2H, m, amide), 7.80 (4H, d, J=8.2 Hz, aromatic), 7.36 (4H, d, J=8.2 Hz, aromatic), 5.45 (2H, s), 4.92 (2H, d, J=12.8 Hz, AB coupling), 4.88 (2H, s), 4.57 (2H, d, J=12.8 Hz, AB coupling), 3.57 (4H, m) 2.70-1.19 (40H, m) 1.45 (6H, s, CH₃), 0.95-0.92 (12H, 2×CH₃).

### Synthesis of Example 24

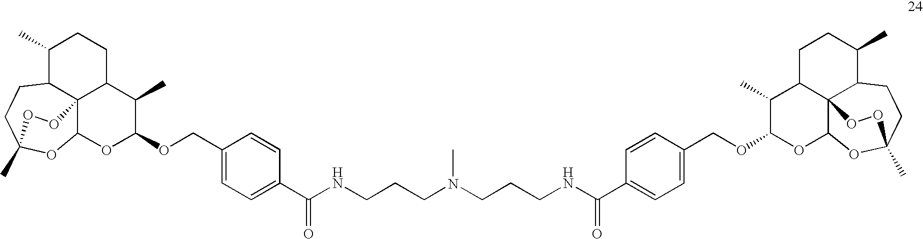

This compound was prepared from compound 15 and 3,3′-diamino-N-methyldipropylamine, using a similar procedure to 4 to give the product in a 27% yield: ¹H NMR (250 MHz, CDCl₃) 7.56 (4H, d, J=8.2 Hz, aromatic), 7.16 (4H, d, J=8.2 Hz, aromatic), 5.24 (2H, s), 4.72 (2H, d, J=13.3 Hz, AB coupling), 4.69 (2H, s), 4.36 (2H, d, J=13.3 Hz, AB coupling), 3.33 (4H, m) 2.47 (2H, m) 2.3 (4H, t, J=6.3 Hz), 2.15 (2H, dt, J=14.2, 3.9 Hz), 2.07 (3H, s, NMe), 1.87-1.03 (26H, m) 1.25 (6H, s, CH₃), 0.76-0.70 (12H, 2×CH₃).

### Synthesis of Example 25

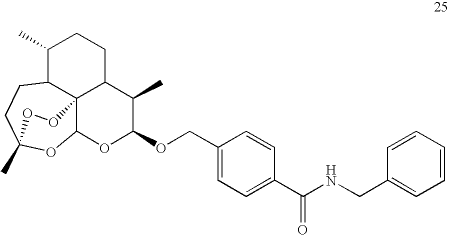

This compound was prepared from compound 15 and benzylamine, using a similar procedure to 4 to give the product in a 20% yield: ¹H NMR (250 MHz, CDCl₃) 7.79 (2H, d, J=8.2 Hz, aromatic), 7.44-7.28 (7H, m, aromatic), 6.46 (1H, m, amide) 5.46 (1H, s), 4.95 (1H, d, J=12.8 Hz, AB coupling), 4.90 (1H, s), 4.66 (2H, d, J=5.63 Hz, benzylic CH₂), 4.57 (1H, d, J=12.8 Hz, AB coupling), 2.68 (1H, m, CH), 2.38 (1H, dt, J=13.54, 3.93 Hz), 2.08-1.24 (10H, m), 1.46 (3H, s, CH₃), 0.97-0.89 (6H, 2×CH₃).

### Synthesis of Example 26

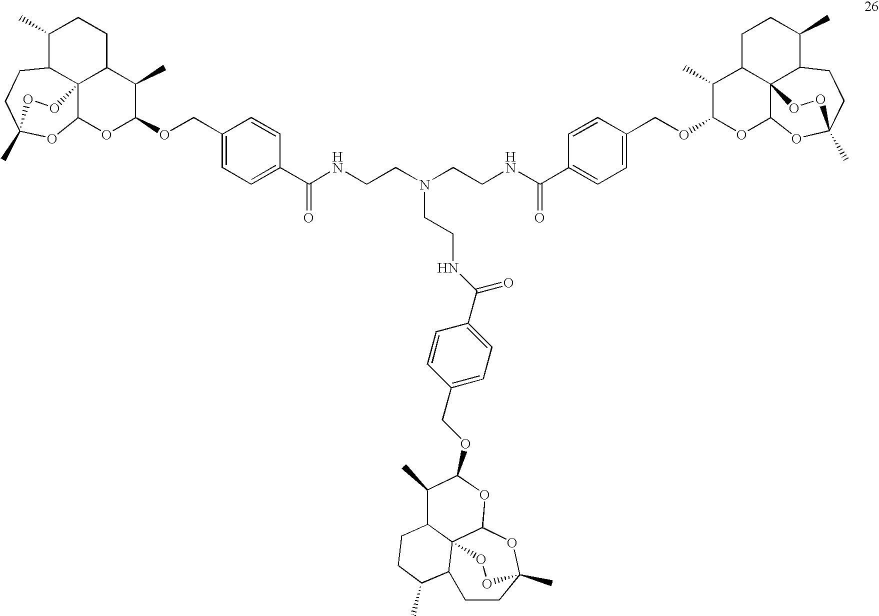

This compound was prepared from compound 15 and tris(2-aminoethyl)amine, using a similar procedure to 4 to give the product in a 13% yield: ¹H NMR (250 MHz, CDCl₃) 7.69 (6H, d, J=8.1 Hz, aromatic), 7.18 (6H, d, J=8.1 Hz, aromatic), 5.43 (3H, s), 4.86 (3H, d, J=12.4 Hz, AB coupling), 4.86 (3H, s), 4.50 (3H, d, J=12.4 Hz, AB coupling), 3.56 (6H, m), 2.77 (6H, m), 2.67 (3H, m) 2.37 (3H, dt, J=13.8, 3.8 Hz), 1.93-1.21 (33H, m) 1.46 (9H, s, CH₃), 0.96-0.93 (18H, 2×CH₃).

### Synthesis of Example 27

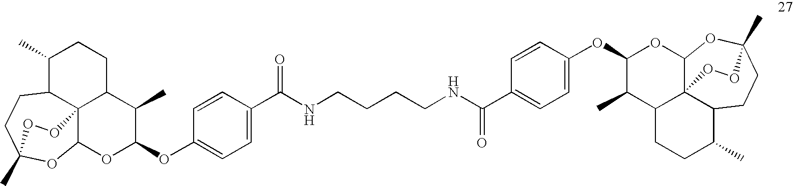

This compound was prepared from compound 17 and 1,4-diaminobutane, using a similar procedure to 4 to give the product in a 17% yield: ¹H NMR (250 MHz, CDCl₃) 7.79 (4H, d, J=8.6 Hz, aromatic), 7.14 (4H, d, J=8.6 Hz, aromatic), 6.66 (2H, m, amide), 5.57 (2H, d, J=3.3 Hz), 5.46 (2H, s), 3.50 (4H, m), 2.83 (2H, m, CH), 2.39 (2H, dt, J=13.9, 3.8 Hz), 2.07-1.22 (24H, m), 1.44 (6H, s, CH₃), 1.04-0.93 (12H, 2×CH₃).

### Synthesis of Example 28

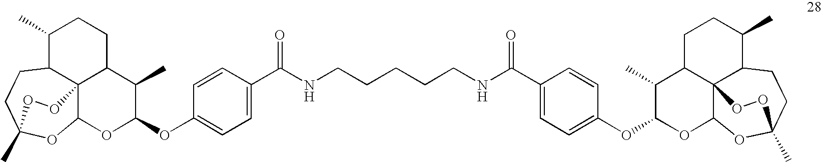

This compound was prepared from compound 17 and 1,5-diaminopentane, using a similar procedure to 4 to give the product in a 25% yield: ¹H NMR (250 MHz, CDCl₃) 7.63 (4H, d, J=8.8 Hz, aromatic), 7.03 (4H, d, J=8.8 Hz, aromatic), 6.14 (2H, m, amide), 5.46 (2H, d, J=3.2 Hz), 5.34 (2H, s), 3.35 (4H, m), 2.71 (2H, m, CH), 2.26 (2H, dt, J=13.6, 4.0 Hz), 1.95-1.11 (26H, m), 1.32 (6H, s, CH₃), 0.93-0.84 (12H, 2×CH₃).

### Synthesis of Example 29

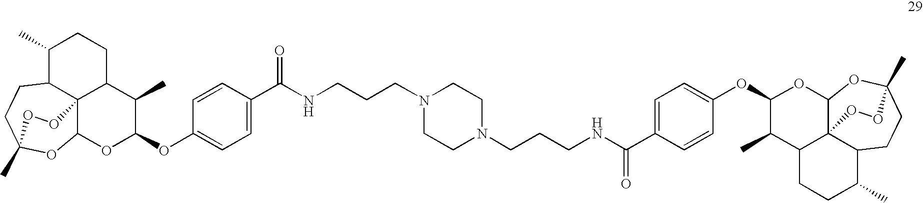

This compound was prepared from compound 17 and 1,4-bis(3-aminopropyl)-piperazine, using a similar procedure to 4 to give the product in a 28% yield: ¹H NMR (250 MHz, CDCl₃) 7.97 (2H, m, amide), 7.79 (4H, d, J=8.7 Hz, aromatic), 7.15 (4H, d, J=8.7 Hz, aromatic), 5.56 (2H, d, J=3.4 Hz), 5.47 (2H, s), 3.58 (4H, m), 2.85 (2H, m, CH), 2.58 (10H, m), 2.40 (2H, dt, J=14.0, 3.8 Hz), 2.08-1.15 (26H, m), 1.44 (6H, s, CH₃), 1.05-0.97 (12H, 2×CH₃).

### Synthesis of Example 30

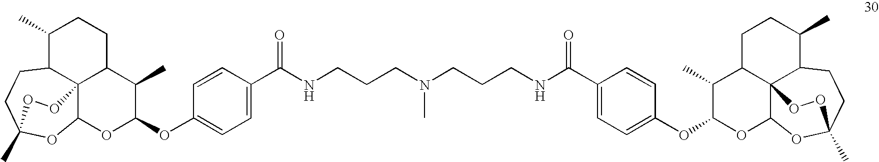

This compound was prepared from compound 17 and 3,3′-diamino-N-methyldipropylamine, using a similar procedure to 4 to give the product in a 17% yield: ¹H NMR (250 MHz, CDCl₃) 7.78 (4H, d, J=8.7 Hz, aromatic), 7.33 (2H, m, amide), 7.15 (4H, d, J=8.7 Hz, aromatic), 5.56 (2H, d, J=3.1 Hz), 5.46 (2H, s), 3.53 (4H, m), 2.83 (2H, m, CH), 2.51 (4H, m), 2.51-1.11 (33H, m), 1.44 (6H, s, CH₃), 1.04-0.96 (12H, 2×CH₃).

### Synthesis of Example 31

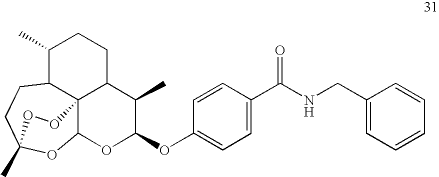

This compound was prepared from compound 17 and benzylamine, using a similar procedure to 4 to give the product in a 54% yield: ¹H NMR (250 MHz, CDCl₃) 7.76 (2H, d, J=8.8 Hz, aromatic), 7.38-7.28 (5H, m, aromatic), 7.16 (2H, d, J=8.8 Hz, aromatic), 6.37 (1H, m, amide), 5.56 (1H, d, J=3.3 Hz), 5.46 (1H, s), 4.65 (2H, d, J=5.6 Hz, benzylic), 2.84 (1H, m, CH), 2.39 (1H, dt, J=13.3, 4.0 Hz), 2.08-1.22 (10H, m), 1.45 (3H, s, CH₃), 1.04-0.96 (6H, 2×CH₃).

### Synthesis of Example 32

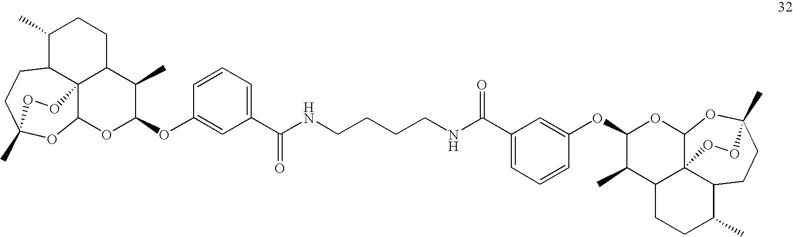

This compound was prepared from compound 16 and 1,4-diaminobutane, using a similar procedure to 4 to give the product in a 23% yield: ¹H NMR (250 MHz, CDCl₃) 7.57-7.21 (8H, m, aromatic), 6.92 (2H, m, amide), 5.60 (2H, d, J=3.2 Hz), 5.48 (2H, s), 3.46 (4H, m), 2.80 (2H, m, CH), 2.37 (2H, dt, J=13.9, 3.6 Hz), 2.05-1.22 (24H, m), 1.38 (6H, s, CH₃), 1.04-0.95 (12H, 2×CH₃).

### Synthesis of Example 33

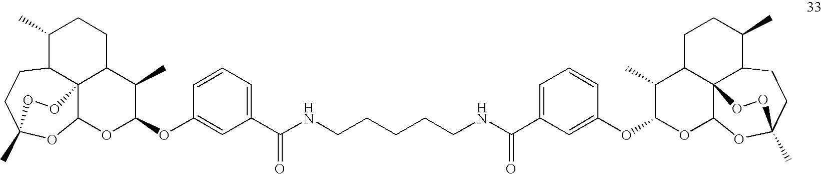

This compound was prepared from compound 16 and 1,5-diaminopentane, using a similar procedure to 4 to give the product in a 28% yield: ¹H NMR (250 MHz, CDCl₃) 7.35-6.98 (8H, m, aromatic), 6.5 (2H, m, amide), 5.39 (2H, d, J=3.2 Hz), 5.27 (2H, s), 3.21 (4H, m), 2.61 (2H, m, CH), 2.17 (2H, dt, J=13.9, 4.0 Hz), 1.85-1.02 (26H, m), 1.19 (6H, s, CH₃), 0.83-0.75 (12H, 2×CH₃).

### Synthesis of Example 34

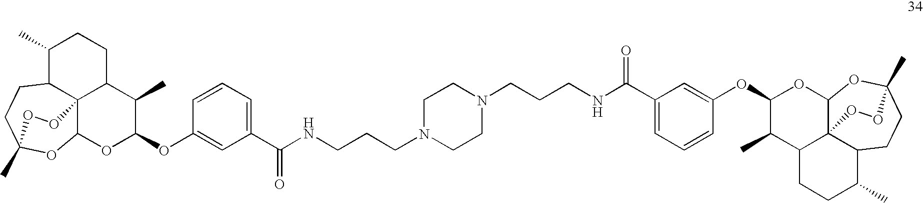

This compound was prepared from compound 16 and 1,4-bis(3-aminopropyl)-piperazine, using a similar procedure to 4 to give the product in a 42% yield: ¹H NMR (250 MHz, CDCl₃) 8.03 (2H, m, amide), 7.55-6.23 (8H, m, aromatic), 5.56 (2H, d, J=3.1 Hz), 5.46 (2H, s), 3.53 (4H, m), 2.81 (2H, m, CH), 2.53-1.20 (42H, m), 1.41 (6H, s, CH₃), 1.03-0.94 (12H, 2×CH₃).

### Synthesis of Example 35

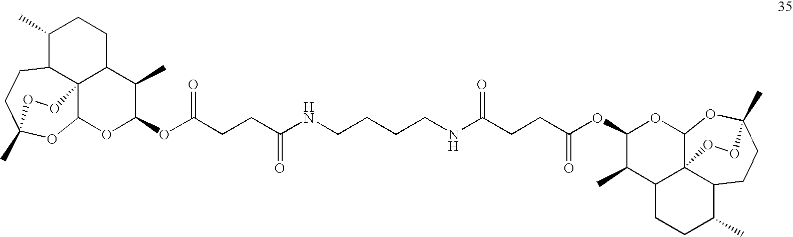

This compound was prepared from compound 18 and 1,4-diaminobutane, using a similar procedure to 4 to give the product in a 14% yield: ¹H NMR (250 MHz, CDCl₃) 6.20 (2H, m, amide), 5.77 (2H, d, J=9.9 Hz), 5.44 (2H, s), 3.25 (4H, m), 2.76 (4H, m, CH), 2.57-1.26 (32H, m), 1.43 (6H, s, CH₃), 0.97 (6H, d, J=5.7 Hz, CH₃), 0.85 (6H, d, J=7.09).

It is to be understood that the present invention is not intended to be restricted to the details of the above examples and embodiments, which are described by way of example only.

1. A compound of the following general formula: 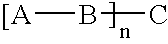
or a pharmaceutically acceptable salt thereof,
in which:
n=an integer of from 1 to 4;
A represents a trioxane-containing residue having the following structure; 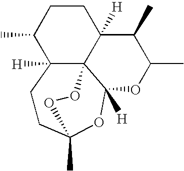
B represents a group having the general formula:
-D-E-F-
in which:
D is linked to A and represents an atom or group selected from the following: 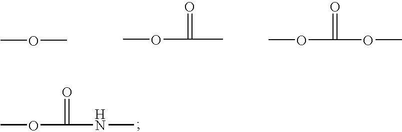
E represents a bivalent, optionally substituted organic radical having at least 3 carbon atoms; and
F is linked to C and represents a group selected from the following: 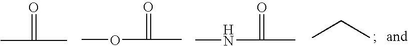
C represents a group containing at least two amino groups.

2. The compound according to claim 1, wherein E comprises an organic radical of 3-50 carbon atoms.

3. The compound according to claim 2, wherein E comprises an organic radical of 3-20 carbon atoms.

4. The compound according to claim 1, wherein E has a substituent selected from primary, secondary and tertiary amines; halogen-containing groups; alcohols and derivatives thereof; and carboxylic acids and derivatives thereof.

5. The compound according to claim 3, wherein E is selected from the group consisting of —CH₂—CH₂—, p-phenylene and pyridine.

6. The compound according to claim 1, wherein C comprises a natural or synthetic polyamine residue of 2-50 carbon atoms.

7. The compound according to claim 1, wherein amino groups, are independently a primary or secondary group, after linking to group F through the same or different amino groups of the polyamine.

8. A compound of the following formula: 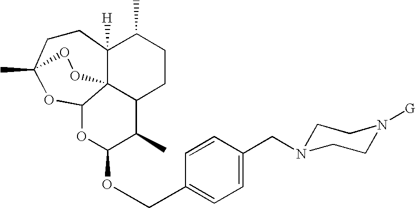
in which:
G represents a substituted aryl group.

9. The compound according to claim 8, wherein the compound comprises a compound of the following formula: 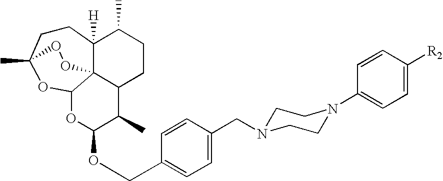
in which:
R₂ is selected from H, NO₂, F, and CF₃.

10. The compound according to claim 8, wherein the compound is:

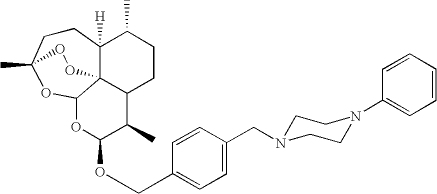

11. The compound according to claim 8, wherein the compound comprises a compound of the following formula: 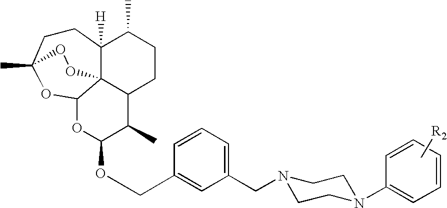
in which:
R₂ is selected from H, pNO₂, p-Cl, m-CF₃, and p-F.

12. The compound according to claim 1, wherein the compound is selected from the following:

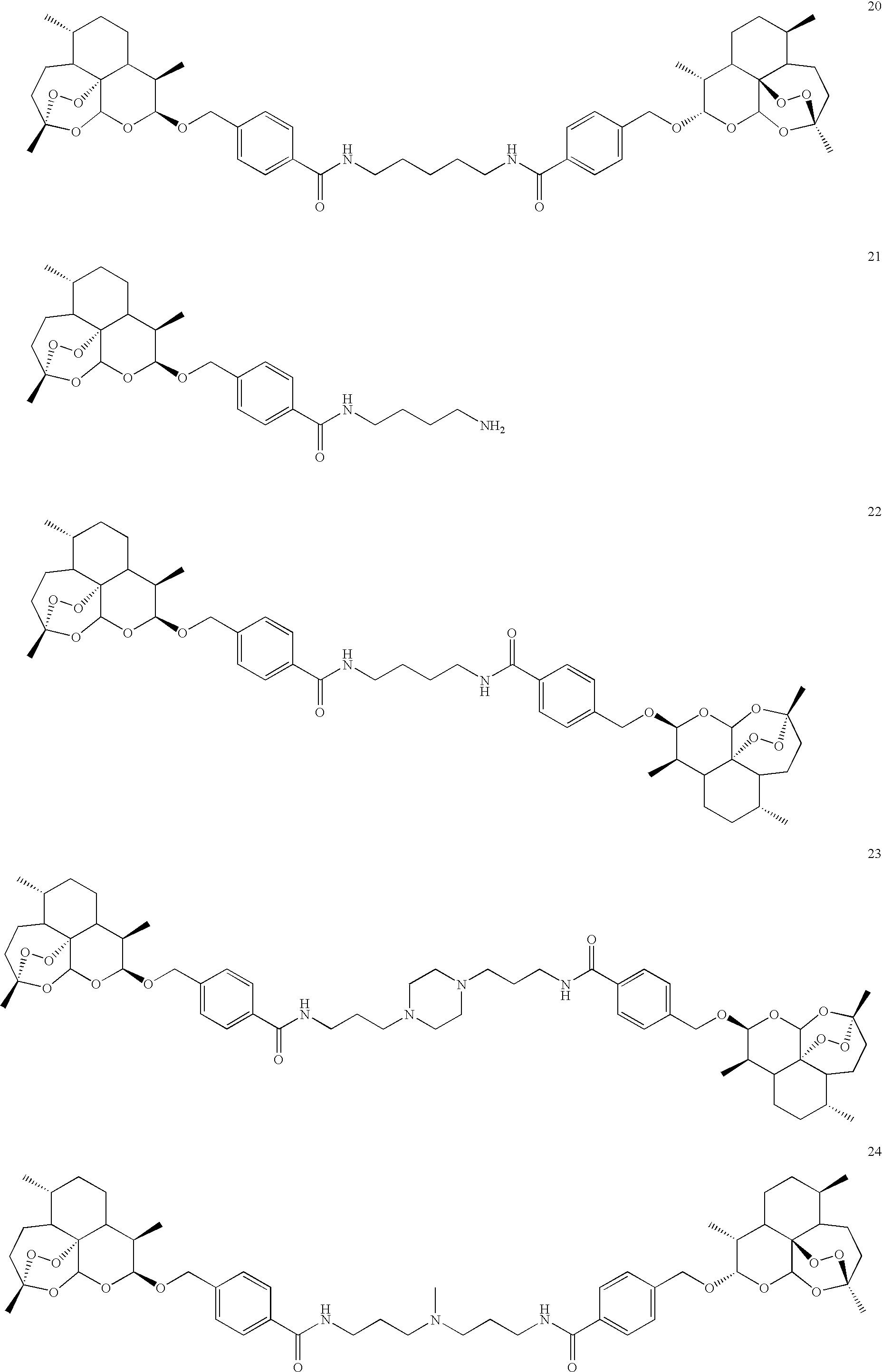

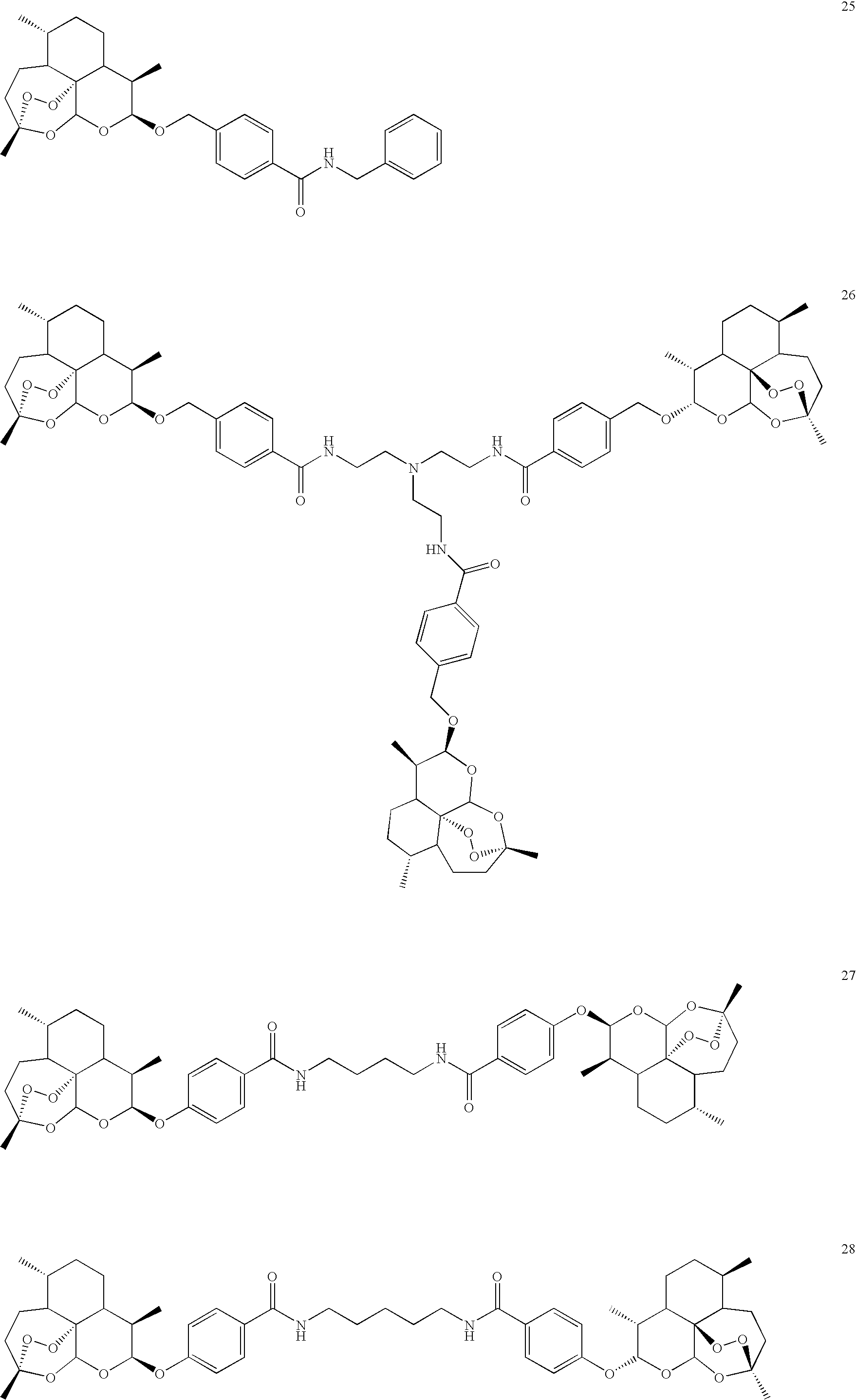

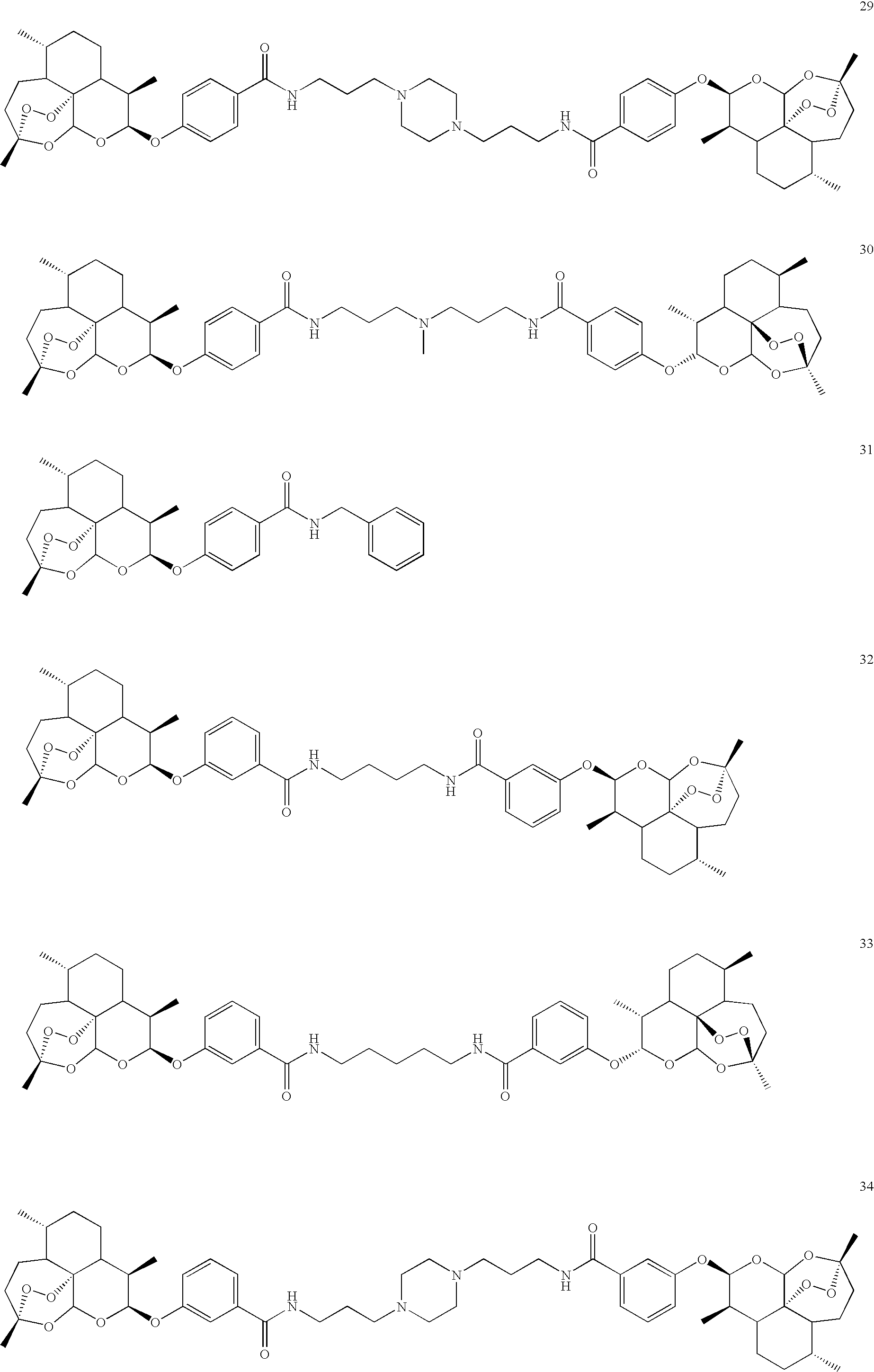

13. A compound of the following formula:

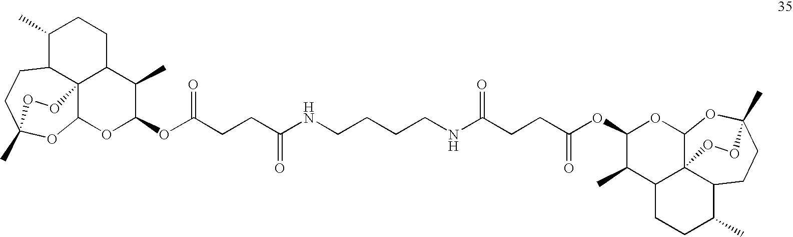

14. A process for the production of a trioxane derivative, comprising the steps of coupling dihydroartemisinin with benzenedimethanol, converting the resultant alcohol into the corresponding sulfonate by treatment with a sulfonyl halide, and reacting said sulfonate with a diamino nucleophile.

15. The process according to claim 14, wherein the resultant alcohol is converted into the corresponding mesylate by treatment with mesyl chloride.

16. The process according to claim 14, wherein the trioxane derivative comprises a compound according to claim 1.

17. A process for the production of a trioxane derivative, comprising the steps of coupling dihydroartemisinin with an alcohol methyl ester, hydrolysing the resultant compound to produce the corresponding carboxylic acid, and coupling said carboxylic acid with a polyamine or amine.

18. The process according to claim 17, wherein coupling of the carboxylic acid and polyamine or amine is carried out at a temperature of between −20° C. and +40° C.

19. A process for the production of a trioxane derivative, comprising the steps of coupling dihydroartemisinin with an alcohol methyl ester, hydrolysing the resultant compound to produce the corresponding carboxylic acid, and coupling said carboxylic acid with a polyamine or amine, wherein the trioxane derivative comprises a compound according to claim 12.

20. A process for the production of a trioxane derivative, comprising the steps of coupling dihydroartemisinin with an anhydride, forming a carboxylic acid, and coupling said carboxylic acid with a polyamine or amine.

21. A process for the production of a trioxane derivative, comprising the steps of coupling dihydroartemisinin with an alcohol methyl ester, hydrolysing the resultant compound to produce the corresponding carboxylic acid, and coupling said carboxylic acid with a polyamine or amine, wherein the trioxane derivative comprises a compound according to claim 13.

22. A compound according to claim 1, or a pharmaceutically acceptable salt thereof, for use as a medicament.

23. The compound according to claim 22, wherein the medicament is a medicament for the treatment of malaria or cancer.

24. A pharmaceutical composition containing a compound according to claim 1, or a pharmaceutically acceptable salt thereof, as an active ingredient.

25. A process comprising use of a compound according to claim 1, or a pharmaceutically acceptable salt thereof, in the manufacture of a medicament for the treatment or prophylaxis of malaria.

26. A process comprising use of a compound according to claim 1, or a pharmaceutically acceptable salt thereof, in the manufacture of a medicament for the treatment of cancer.

27. A product containing a first compound according to claim 1, or a pharmaceutically acceptable salt thereof, a second, iron-containing, compound as a combined preparation for simultaneous, separate or sequential use in the treatment of cancers.

28. The product according to claim 27, wherein the first and second compounds are for use sequentially, the second, iron-containing, compound being used first.

29. The product according to claim 27, wherein the second, iron-containing, compound is an iron salt selected from ferrous fumarate, ferrous sulphate, ferrous carbonate, ferrous citrate, ferrous gluconate, ferrous lactate and ferrous maleate.

30. The product according to claim 27, wherein the second, iron-containing, compound is an iron complex selected from ferrocholinate, ferroglycine sulphate, dextran iron complex, peptonized iron, iron sorbitex, saccharated iron, iron complexed with an iron binding protein, and iron complexed with a glycoprotein.

31. The product according to claim 27, wherein the product further comprises one or more other agents known to be useful in the treatment of tumours.

32. The product according to claim 31, wherein the one or more other agents are selected from androgen inhibitors, antiestrogens, antimetabolites and cytotoxic agents.

33. A method of treatment of malaria which comprises administering to an animal in need of such treatment a therapeutically effective amount of a compound according to claim 1, or a pharmaceutically acceptable salt thereof.

34. The method of treatment according to claim 33, wherein the compound is administered in an amount of 50 to 1000 mg.

35. A method of treatment of cancer which comprises administering to an animal in need of such treatment a therapeutically effective amount of a first compound according to claim 1, or a pharmaceutically acceptable salt thereof.

36. The method of treatment according to claim 35, wherein the method further comprises the simultaneous, separate or sequential administration to the said animal of an effective amount of a second, iron-containing, compound.

37. The method according to claim 35, wherein the first compound is administered at a dosage within the range of 0.5 to 300 mg/kg body weight.

38. The method according to claim 37, wherein the first compound is administered at a dosage within the range of 1 to 50 mg/kg body weight.

39. The method according to claim 35, wherein second, iron-containing, compound is administered at a dosage within the range of 0.01 to 1000 mg iron/kg body weight.
